SEQUENCE LISTING (8 sequences)

SEQ ID NO: 1 - DNA, 2009 bp, Arabidopsis thaliana
  gene (1)..(2009)
aataaataaataaaaatgtagccaattttgtatataaatgagttctgattatctatataaataccaaagccatctgctcatctttaagccccacaggccaaaagacactctccttttgtttatctctctcttctcttcacttactttgctttgatccgccatggctacgaaactcgaaagctccttaatctttgcccttttgtccaaatgcagcgttctaagccaaaccaaccttgccttctccctcctcgccgtcacaatcatctggctcgccatatctctcttcttatggacctatcccggtggacctgcttgggggaaatacctcttcggccggttaatatccggttcatacaaaaccggaaacgttattcccggtccaaaaggcttccctttggttggaagcatgtcactcatgtcaagcactctagctcaccgacgaatcgctgatgcagctgagaaattcggagccaagaggctcatggctttcagcttaggagagactcgcgtgatcgtcacgtgcaatcccgacgtagcgaaagagattctgaatagcccggtttttgctgatcgaccggttaaagaatcggcttactcactgatgtttaacagagcaattggttttgcaccacacggtgtttactggcgaacgcttcgccgtatcgcttcgaaccatctctttagtacaaaacaaatcagaagagccgagacgcaacgacgagtgatctcaagccagatggttgagtttcttgaaaaacagagtagtaacgaaccctgttttgttcgtgagttgcttaaaacggcgtcgcttaacaacatgatgtgctctgtattcggacaagagtatgagcttgaaaaaaaccatgttgagttacgtgaaatggtcgaagaaggttatgatttgctcggaacgttgaattggactgatcaccttccttggctatcggagtttgatcctcaaagactccggtctagatgttccacactcgtaccaaaggtaaaccggtttgtatcccggattatatccgaacaccgtaatcaaaccggtgatttgcctcgtgatttcgtcgacgttttgctctccctccatggttcagataaattatccgacccggacataatcgccgttctttgggtatgcacaccatttatttgattaattattcttaattatatttgttgaaaattgcttaggattatttagattaaaacatgaaatttgagactcaatgtgacgtgttgtggaataattaaagcattagaagttttttgtttgacatcaaattagtaaattttagattttataacagtttctataaaaagtaaaaagtactaaaatttttgagttattattacaggagatgatattcagaggaacagacacagttgcggtcttaatcgagtggatcctcgctaggatggtccttcatccagatatgcaatcaacggtacaaaacgagctggatcaagtagtcgggaaatcaagagccctagatgaatctgacttggcttcacttccatatctaacggctgtggtgaaagaagtattgaggcttcatcctccaggcccacttctatcatgggcccgtttggccataacagacacgatcgttgatggtcgtcttgttccggcagggaccacagcaatggtgaacatgtgggccgtatcgcatgatccacacgtgtgggttgatcctttggagtttaaacctgagaggttcgtggcaaaagaaggtgaggtggagttttcggttcttgggtcggatttgagacttgcacctttcgggtcgggtcgtcggatttgccccgggaagaatcttggttttactaccgttatgttttggacggcgatgatgttacatgagtttgaatggggaccgtccgatggtaacggcgttgacttatctgagaaactgaggctttcttgcgagatggctaatcctcttcctgctaaattgcgccgtaggcgcagttaaaaaaaagaagctcatatgagaattagagatttt

SEQ ID NO: 2 - DNA, 1593 bp, Arabidopsis thaliana
  CDS (1)..(1593)
atggctacgaaactcgaaagctccttaatctttgcccttttgtccaaatgcagcgttctaagccaaaccaaccttgccttctccctcctcgccgtcacaatcatctggctcgccatatctctcttcttatggacctatcccggtggacctgcttgggggaaatacctcttcggccggttaatatccggttcatacaaaaccggaaacgttattcccggtccaaaaggcttccctttggttggaagcatgtcactcatgtcaagcactctagctcaccgacgaatcgctgatgcagctgagaaattcggagccaagaggctcatggctttcagcttaggagagactcgcgtgatcgtcacgtgcaatcccgacgtagcgaaagagattctgaatagcccggtttttgctgatcgaccggttaaagaatcggcttactcactgatgtttaacagagcaattggttttgcaccacacggtgtttactggcgaacgcttcgccgtatcgcttcgaaccatctctttagtacaaaacaaatcagaagagccgagacgcaacgacgagtgatctcaagccagatggttgagtttcttgaaaaacagagtagtaacgaaccctgttttgttcgtgagttgcttaaaacggcgtcgcttaacaacatgatgtgctctgtattcggacaagagtatgagcttgaaaaaaaccatgttgagttacgtgaaatggtcgaagaaggttatgatttgctcggaacgttgaattggactgatcaccttccttggctatcggagtttgatcctcaaagactccggtctagatgttccacactcgtaccaaaggtaaaccggtttgtatcccggattatatccgaacaccgtaatcaaaccggtgatttgcctcgtgatttcgtcgacgttttgctctccctccatggttcagataaattatccgacccggacataatcgccgttctttgggagatgatattcagaggaacagacacagttgcggtcttaatcgagtggatcctcgctaggatggtccttcatccagatatgcaatcaacggtacaaaacgagctggatcaagtagtcgggaaatcaagagccctagatgaatctgacttggcttcacttccatatctaacggctgtggtgaaagaagtattgaggcttcatcctccaggcccacttctatcatgggcccgtttggccataacagacacgatcgttgatggtcgtcttgttccggcagggaccacagcaatggtgaacatgtgggccgtatcgcatgatccacacgtgtgggttgatcctttggagtttaaacctgagaggttcgtggcaaaagaaggtgaggtggagttttcggttcttgggtcggatttgagacttgcacctttcgggtcgggtcgtcggatttgccccgggaagaatcttggttttactaccgttatgttttggacggcgatgatgttacatgagtttgaatggggaccgtccgatggtaacggcgttgacttatctgagaaactgaggctttcttgcgagatggctaatcctcttcctgctaaattgcgccgtaggcgcagttaa

SEQ ID NO: 3 - protein, 530 aa, Arabidopsis thaliana
MATKLESSLIFALLSKCSVLSQTNLAFSLLAVTIIWLAISLFLWTYPGGPAWGKYLFGRLISGSYKTGNVIPGPKGFPLVGSMSLMSSTLAHRRIADAAEKFGAKRLMAFSLGETRVIVTCNPDVAKEILNSPVFADRPVKESAYSLMFNRAIGFAPHGVYWRTLRRIASNHLFSTKQIRRAETQRRVISSQMVEFLEKQSSNEPCFVRELLKTASLNNMMCSVFGQEYELEKNHVELREMVEEGYDLLGTLNWTDHLPWLSEFDPQRLRSRCSTLVPKVNRFVSRIISEHRNQTGDLPRDFVDVLLSLHGSDKLSDPDIIAVLWEMIFRGTDTVAVLIEWILARMVLHPDMQSTVQNELDQVVGKSRALDESDLASLPYLTAVVKEVLRLHPPGPLLSWARLAITDTIVDGRLVPAGTTAMVNMWAVSHDPHVWVDPLEFKPERFVAKEGEVEFSVLGSDLRLAPFGSGRRICPGKNLGFTTVMFWTAMMLHEFEWGPSDGNGVDLSEKLRLSCEMANPLPAKLRRRRS

SEQ ID NO: 4 - DNA, 15 bp, artificial sequence
  primer_bind (1)..(15): pBR322 primer
attatcacattaacc

SEQ ID NO: 5 - DNA, 26 bp, artificial sequence
  primer_bind (1)..(26): P45F151 - P450 cDNA primer
ttgatccgccatggctacgaaactcg

SEQ ID NO: 6 - DNA, 26 bp, artificial sequence
  primer_bind (1)..(26): P45R1976 - P450 cDNA primer
ttaactgcgcctacggcgcaatttag

SEQ ID NO: 7 - DNA, 34 bp, artificial sequence
  primer_bind (1)..(34): KpnI/EcoRI P450F - P450 cDNA primer
gggtaccgaattcatggctacgaaactcgaaagc

SEQ ID NO: 8 - DNA, 36 bp, artificial sequence
  primer_bind (1)..(36): HindIII/SacI P450R - P450 cDNA primer
gcataagcttgagctcttaactgcgcctacggcgca

(19) United States
(12) Patent Application Publication
(10) Pub. No.: US 2003/0092014 A1
(43) Pub. Date: May 15, 2003

(54) Nucleic acid molecules and polypeptides for catabolism of abscisic acid

(76) Inventors: John R. Coleman, Toronto (CA); Judith Jebanathirajah, Scarborough (CA); Fernando Ferreira, Mississauga (CA)

Correspondence Address: Patrick J. Kelly, Ph.D., Synnestvedt & Lechner LLP, 2600 Aramark Tower, 1101 Market Street, Philadelphia, PA, 19107-2950, US

(21) Appl. No.: 10/022,025

(22) Filed: Dec. 13, 2001

Related U.S. Application Data
(60) Provisional application No. 60/254,819, filed on Dec. 13, 2000.

(51) Int. Cl. C12Q 1/68; C07H 21/04; C12N 9/02; C12P 21/02; C12N 5/06

(52) U.S. Cl. 435/6; 435/69.1; 435/320.1; 435/189; 435/325; 536/23.2

(57) ABSTRACT

The invention includes ABACP nucleic acid molecules and polypeptides involved in modulating seed development, stomate regulation and plant adaptation to environmental stresses such as drought, cold and a high carbon dioxide environment.

### RELATED APPLICATION

This application is based on and claims priority of U.S. Provisional Application No. 60/254,819, filed Dec. 13, 2000.

### FIELD OF THE INVENTION

The invention relates to nucleic acid molecules and polypeptides involved in plant metabolism, and more particularly in modulating seed development, stomate regulation and plant adaptation to environmental stresses.

### BACKGROUND OF THE INVENTION

Abscisic acid (ABA) is a phytohormone that regulates plant development and metabolism. It is involved in seed development, stomate regulation and plant adaptation to environmental stresses such as drought, cold and other stressful environments. It has been proposed that (+)-ABA 8′-hydroxylase, a putative cytochrome P450, is involved in ABA regulation through ABA catabolism. However, to date, no one has been able to isolate and sequence the (+)-ABA 8′-hydroxylase gene or protein. In fact, the protein has not even been purified to homogeneity as identified as a single band by protein gel electrophoresis. Some preliminary functional characterization has been achieved, but this information is inadequate to allow others to modulate the (+)-ABA 8′-hydroxylase gene and protein. There are many reasons for the problems in isolating the gene and protein, including the very low levels of gene expression and resulting enzyme activity, the instability of enzyme activity in plant extracts, and its association with membranes and cofactors which appear to be required for catalytic activity. Without the gene and protein sequences, it is impossible to design rational strategies for control of ABA levels by modulating the (+)-ABA 8′-hydroxylase gene and protein. There is a need to identify these sequences in order to identify methods to control seed development, stomate regulation and plant adaptation to environmental stresses. Protein sequence information is essential for the elucidation of protein structure and the ultimate design of chemical effectors that may modulate activity in plants. There is also a need for transgenic plants which overexpress these polypeptides and plants in which gene expression is reduced or blocked. In transgenic plants, using well described genetic technologies, it will be possible to control levels of expression in specific locations in plants and over a specific developmental time point or in response to a particular abiotic or biotic stress event. In this way it will be possible to modulate the levels of ABA in a specific and desirable fashion.

The roles of Cytochrome P450s (or heme monoxygenases) in plant metabolism are poorly defined. Cytochrome P450s are a superfamily of enzymes found in both prokaryotes and eukaryotes and are involved in biosynthesis or degradation of both exogenous and endogenous chemicals including steroids, fatty acids, and secondary metabolites. Common to all P450s is an iron-protoporphyrin IX complex, which is the donor of the reactive oxygen atom during substrate oxidation and a cysteine residue, which is an axial ligand of the iron in this prosthetic group ¹⁹. The core containing the heme-binding site is highly conserved whereas the regions associated with substrate recognition and redox partner binding are highly variable. This variability in sequence confers the P450s with regio and/or stereo-product selectivity. The predicted number of monooxygenase genes in Arabidopsis is 300-350 and currently there is EST evidence for approximately 204 genes. Phylogentic studies have shown that plant, fungi and animal P450s arose from a single ancestor that had a variant of CYP51 ²⁰. Given this information, it is interesting to note that yeast have 2 P450 genes, C. elegans has 80 P450s and mammals are predicted to have 50-80 P450s. The number of P450s found in Arabidopsis shows an immense investment in biochemical complexity which has been engaged in many ways. Complex biochemical pathways using various monooxygenases have been shown to produce toxic alkaloids and phytoalexins for defence against herbivory and pathogens, and other products include pigments and aromatics made to attract pollinators. The number of P450s predicted for Arabidopsis appears to be representative of most plants, indeed it seems as if Arabidopsis is missing some families of P450s which are found in other plants ²⁰. According to the UPGMA tree of plant P450s CYP78 falls into clan A between CYP79A1 and CYP99. Clusters of P450s are not from organisms that fshare a common ancestor but they probably represent genes that diverged from a single ancestral sequence.

Most P450 catalyzed reactions are NADPH and O₂ dependent hydroxylations, however they are also known to perform N-dealkylation, O-dealkylation, oxidative deamination, oxidatiove dehalogenation and other reactions. The reaction requires two reducing equivalents which are usually delivered to the P450 via a NADPH reductase when both substrate and O₂ are bound to the P450. Most P450 reactions proceed with the stoichiometry characteristic of monooxygenases. Several plant P450 have been cloned from Arabidopsis and other plants recently but their roles in plant metabolism are still not well understood.

### SUMMARY OF THE INVENTION

The invention relates to cytochrome P450 nucleic acid molecules and polypeptides involved in catabolism of ABA, and more particularly in modulating seed development, stomate regulation and plant adaptation to environmental stresses such as drought and cold.

The invention relates to an isolated nucleic acid molecule encoding an ABACP polypeptide, or a fragment of an ABACP polypeptide having ABACP polypeptide activity. The polypeptide catabolizes ABA. The polypeptide preferably comprises a (+)-ABA 8′hydroxylase.

Another aspect of the invention relates to an isolated nucleic acid molecule encoding an ABACP polypeptide, a fragment of an ABACP polypeptide having ABACP activity, or a polypeptide having ABACP activity, comprising a nucleic acid molecule selected from the group consisting of:

(a) a nucleic acid molecule that hybridizes to a nucleic acid molecule consisting of [SEQ ID NO:1 or 2], or a complement thereof under low, moderate or high stringency hybridization conditions wherein the nucleic acid molecule encodes an ABACP polypeptide or a polypeptide having ABACP activity;

(b) a nucleic acid molecule degenerate with respect to (a), wherein the nucleic molecule encodes an ABACP polypeptide or a polypeptide having ABACP activity.

The hybridization conditions optionally comprise low stringency conditions of 1×SSC, 0.1% SDS at 50° C. or high stringency conditions of 0.1×SSC, 0.1% SDS at 65° C.

Another aspect of the invention relates to an isolated nucleic acid molecule encoding an ABACP polypeptide, a fragment of an ABACP polypeptide having ABACP activity, or a polypeptide having ABACP activity, comprising a nucleic acid molecule selected from the group consisting of:

(a) the nucleic acid molecule of the coding strand shown in [SEQ ID NO:1 or 2], or a complement thereof;

(b) a nucleic acid molecule encoding the same amino acid sequence as a nucleotide sequence of (a); and

(c) a nucleic acid molecule having at least 17% identity with the nucleotide sequence of (a) and which encodes an ABACP polypeptide or a polypeptide having ABACP activity.

The ABACP polypeptide may comprise a (+)-ABA 8′hydroxylase polypeptide. The nucleic acid molecule optionally comprises all or part of a nucleotide sequence shown in [SEQ ID NO:1 or 2] or a complement thereof. The nucleic acid molecule may consist of the nucleotide sequence shown in [SEQ ID NO:1 or 2] or a complement thereof. The invention also relates to a (+)-ABA 8′hydroxylase nucleic acid molecule isolated from Arabidopsis thaliana, or a fragment thereof.

Another aspect of the invention is a recombinant nucleic acid molecule comprising a nucleic acid molecule of the invention and a constitutive promoter sequence or an inducible promoter sequence, operatively linked so that the promoter enhances transcription of the nucleic acid molecule in a host cell. The molecule optionally comprises genomic DNA, cDNA or RNA. The nucleic acid molecule is optionally chemically synthesized. Another variation includes an isolated nucleic acid molecule comprising a nucleic acid molecule selected from the group consisting of 8 to 10 nucleotides of the nucleic acid molecule of claim 6, 11 to 25 nucleotides of the nucleic acid molecule of claim 6 and 26 to 50 nucleotides of the nucleic acid molecule of claim 10. The nucleic acid molecule of the invention optionally comprises at least 30 consecutive nucleotides of [SEQ ID NO:1 or 2] or a complement thereof.

The invention also includes a vector comprising a nucleic acid molecule of the invention. The vector may comprise a promoter selected from the group consisting of a super promoter, a 35S promoter of cauliflower mosaic virus, a chemical inducible promoter, a copper-inducible promoter, a steroid-inducible promoter and a tissue-specific promoter. The invention also includes a host cell comprising the recombinant nucleic acid molecule, vector or host cell (or progeny thereof) of the invention. The host cell is preferably selected from the group consisting of a fungal cell, a yeast cell, a bacterial cell, a microorganism cell and a plant cell.

The invention also includes a plant, a plant part, a seed, a plant cell or progeny thereof comprising the recombinant nucleic acid molecule or the vector of the invention. The plant part preferably comprises all or part of a leaf, a flower, a stem, a root or a tuber. The plant, plant part, seed or plant cell is of a species is preferably selected from the group consisting of alfalfa, almond, apple, apricot, arabidopsis, artichoke, atriplex, avocado, barley, beet, birch, brassica, cabbage, cacao, cantalope, carnations, castorbean, caulifower, celery, clover, coffee, corn, cotton, cucumber, garlic, grape, grapefruit, hemp, hops, lettuce, maple, melon, mustard, oak, oat, olive, onion, orange, pea, peach, pear, pepper, pine, plum, poplar, potato, prune, radish, rice, roses, rye, sorghum, soybean, spinach, squash, strawberries, sunflower, tobacco, tomato, wheat. The plant is preferably a dicot plant or a monocot.

The invention includes an isolated polypeptide encoded by and/or produced from the nucleic acid molecule or vector of the invention. The invention includes an isolated ABACP polypeptide or a fragment thereof having ABACP activity. An isolated polypeptide of the invention optionally has an amino acid sequence which comprises at least ten consecutive residues of [SEQ ID NO:3]. The invention also includes an isolated immunogenic polypeptide, the amino acid sequence of which comprises at least 8 consecutive residues of [SEQ ID NO:3]. The invention includes an isolated polypeptide, the amino acid sequence of which comprises residues 52 to 147, 211 to 228 and 468 to 477 of [SEQ ID NO:3]. The polypeptide of the invention may comprise all or part of an amino acid sequence in [SEQ ID NO:3]. The invention includes a polypeptide fragment of the ABACP polypeptide of the invention, or a peptide mimetic of the ABACP polypeptide. The polypeptide fragment may consist of at least 20 amino acids, which fragment has ABACP activity. A fragment or peptide mimetic of the invention is preferably capable of being bound by an antibody to the polypeptide of the invention. The polypeptide of the invention is optionally recombinantly produced.

The invention includes an isolated and purified polypeptide comprising the amino acid sequence of an ABACP polypeptide, wherein the polypeptide is encoded by a nucleic acid molecule that hybridizes under moderate or stringent conditions to a nucleic acid molecule in [SEQ ID NO:1 or 2], a degenerate form thereof or a complement. The invention includes a polypeptide comprising a sequence having greater than 70% sequence identity to a polypeptide of the invention. The polypeptide preferably comprises an ABACP polypeptide. The polypeptide is optionally isolated from Arabidopsis thaliana. The polypeptide preferably comprises a membrane spanning anchor domain including at least 70% sequence identity to the membrane spanning anchor domain of [SEQ ID NO.:32] and/or an heme binding domain including at least 70% sequence identity to the heme binding domain of [SEQ ID NO.:3].

The invention further includes an isolated nucleic acid molecule encoding a polypeptide of the invention. The invention also includes an antibody directed against a polypeptide of the invention. The antibody is preferably a monoclonal antibody or a polyclonal antibody.

The invention includes a nucleic acid molecule comprising a DNA sequence encoding an antisense RNA molecule operably linked to a promoter, the promoter functioning in a plant cell, the antisense RNA molecule complementary to a portion of the coding sequence for a polypeptide having enzymatic activity in the oxidation of ABA in plant cells and wherein said polypeptide comprises an ABACP polypeptide. The ABACP polypeptide preferably comprises ABACP1.

### BRIEF DESCRIPTION OF THE DRAWINGS

Preferred embodiments will be described in relation to the drawings in which:

FIG. 1 represents [SEQ ID NO:1]. In a preferred embodiment, this sequence represents the Arabidopsis thaliana (Col) 5′-3′ genomic sequence of P450 cyp78A6 (ABACP1). Start codon ATG and stop codon TAA bolded and underlined. Position of single internal intron indicated by underlining.

FIG. 2 represents [SEQ ID NO:2]. In a preferred embodiment, this sequence represents the cDNA sequence covering the coding region of P450 cyp 78A6 (ABACP1).

FIG. 3 represents [SEQ ID NO:3]. In a preferred embodiment, this sequence represents the ABACP1 amino acid sequence for the coding region obtained from cDNA sequence analysis.

FIG. 4 shows a typical hydroxylation reaction carried out by P450 monooxygenases.

FIG. 5 shows the catabolism and anabolism of ABA. The arrow marked 8′hydroxylase is the reaction carried out by CNR2.

### DETAILED DESCRIPTION OF THE INVENTION

In this application, the term “isolated nucleic acid” refers to a nucleic acid the structure of which is not identical to that of any naturally occurring nucleic acid or to that of any fragment of a naturally occurring genomic nucleic acid spanning more than three separate genes. The term therefore covers, for example, (a) DNA which has the sequence of part of a naturally occurring genomic DNA molecules; (b) a nucleic acid incorporated into a vector or into the genomic DNA of a prokaryote or eukaryote, respectively, in a manner such that the resulting molecule is not identical to any naturally occurring vector or genomic DNA; (c) a separate molecule such as cDNA, a genomic fragment, a fragment produced by reverse transcription of polyA RNA which can be amplified by PCR, or a restriction fragment; and (c) a recombinant nucleotide sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such as a cDNA or genomic DNA library.

In this study, we report the isolation and preliminary characterization of ABACP nucleic acid molecules and polypeptides, and in particular (+)-ABA 8′hydroxylase (ABACP1) polypeptide cDNA which encodes a cytochrome P450 for ABA catabolism in Arabidopsis thaliana. ABACP polypeptides represent a novel class of P450's in higher plants.

The invention includes methods of upregulating and downregulating ABACP levels in plants. The invention also includes transgenic plants overexpressing ABACP, preferably ABACP1.

The transformed plants overexpressing ABACP are useful because they have increased stomate opening and increased gas exchange (increased stomatal conductance). Transformation of seeds also allows control over seed germination. For example, synchronous or early germination may be obtained. In seeds, the gene is preferably expressed under the control of an inducible promoter such as a temperature or chemical sensitive promoter.

ABACP may be down-regulated to increase plant drought and cold tolerance. Down regulation also allows plants to tolerate high carbon dioxide environments.

Characterization of ABACP

The nucleic acid molecules and polypeptides of the invention were identified following isolation of a mutant plant (cnr 2-1)with a lesion in a cytochrome P450 monooxygenase. The P450 monooxygenase (ABACP1) initiates the initial stage of ABA catabolism and in planta results in the production of inactive compounds

The loss-of-function mutants showed the function of the polypeptide. The cnr 2-1 mutant contains a lesion involved in ABA metabolism. The mutant exhibits lower rates of stomatal conductance as determined by gas exchange analysis, reduced rates of water loss in excised rosettes, reduced rates of water loss from whole plants grown in soil, and reduced stomatal apertures as seen in the SEM analysis of leaf tissue. The presence of ABA, synthesized under water stress conditions causes changes in ion channel activities, which subsequently results in a loss of turgor in guard cells. This loss of turgor results in a reduction in stomatal aperture limiting water loss from the plant ²¹. The reduced stomatal apertures and concomitant reduced levels of conductance explain the lack of high CO₂ sensitivity, as intracellular levels of CO₂ would be significantly lower than that achieved in the wild type plants when exposed to 3000 ppm. The mutant also displays hypersensitivity to exogenous ABA during germination assays and is hyperdormant, which is explained by the presence of elevated endogenous levels of ABA in the seed. The increased sensitivity of era 1 to exogenous 0.3 mM ABA in comparison to cnr 2-1, also shows that a lesion in a signal transduction pathway ¹⁸ affects germination more than a biochemical lesion. If other ABA degradative pathways exist, they are minor degradative pathways compared with the 8′ ABA hydroxylase pathway ¹⁸.

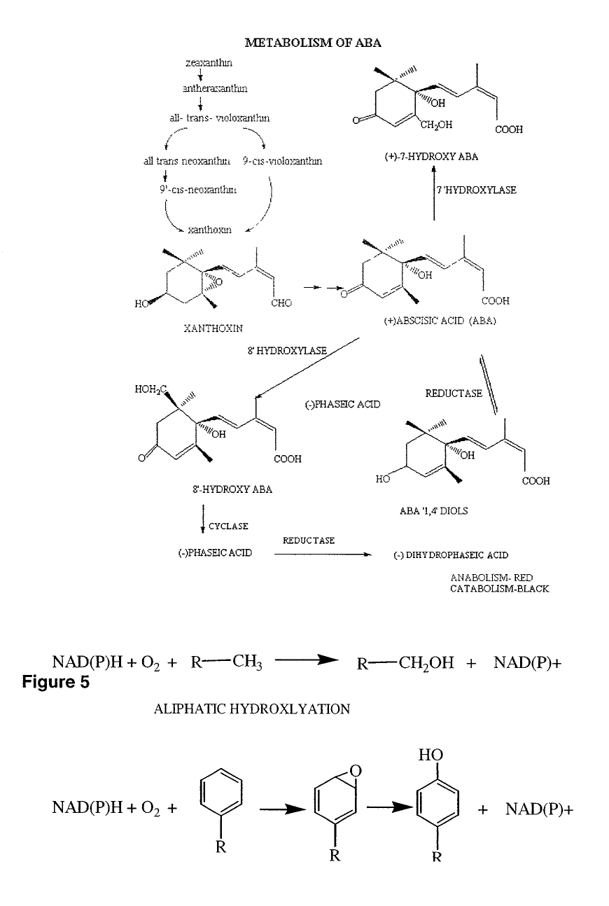

The increased levels of ABA measured in leaf tissue of well watered cnr 2-1 plants, and the high levels and slower turnover of ABA in rehydrated leaf tissue of the mutant again show that this P450 monooxygenase is involved in ABA metabolism. The catabolism of (+)ABA shows the characteristic requirement for NADPH and molecular oxygen observed for a P450 monoxygenase. The 8′ ABA hydroxylase is also inhibited by CO and the inhibition is reversible by light. FIG. 5 shows the catabolism and anabolism of ABA. The arrow marked 8′hydroxylase is the reaction carried out by CNR2.

Nucleic Acid Molecules and Polypeptides

The invention relates to ABACP nucleic acid molecules and polypeptides which are involved in modulating seed development, stomate regulation and plant adaptation to environmental stresses such as drought and cold. These polypeptides preferably include a heme binding domain, an N-terminus hydrophobic membrane anchoring region, and a hinge region domain. The ABACP nucleic acid molecules which encode ABACP polypeptides are particularly useful for producing transgenic plants.

The ABACP nucleic acid molecules and polypeptides, as well as their role in plants were not known before this invention. The ability of these compounds to modulate seed development, stomatal conductance and plant adaptation to environmental stresses such as drought and cold was unknown.

All nucleotides and polypeptides which are suitable for use in the methods of the invention, such as the preparation of transgenic host cells or transgenic plants, are included within the scope of the invention. Genomic clones or cDNA clones are preferred for preparation of transgenic cells and plants.

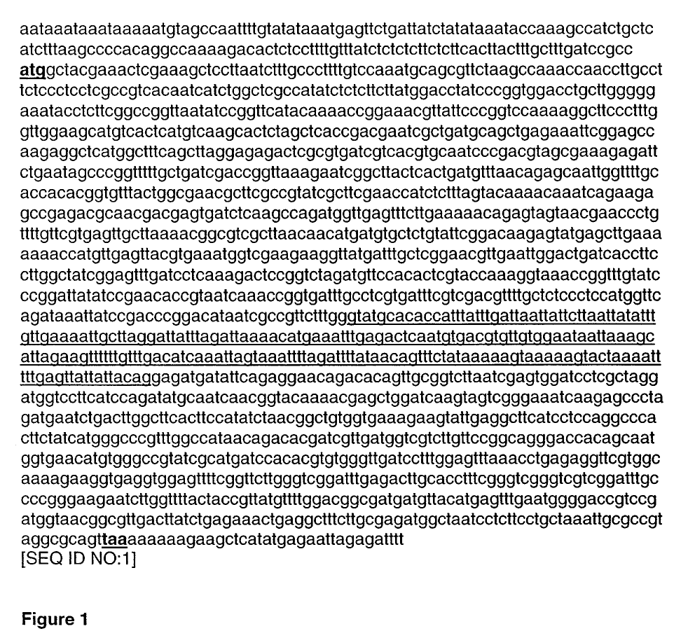

In a preferred embodiment, the invention relates to a cDNA encoding ABACP polypeptides from Arabidopsis thaliana. Preferred sequences and the corresponding amino acid sequence are presented in FIGS. 1-3. The invention also includes splice variants of the nucleic acid molecules as well as polypeptides produced from the molecules.

Characterization of Nucleic Acid Molecules and Polypeptides

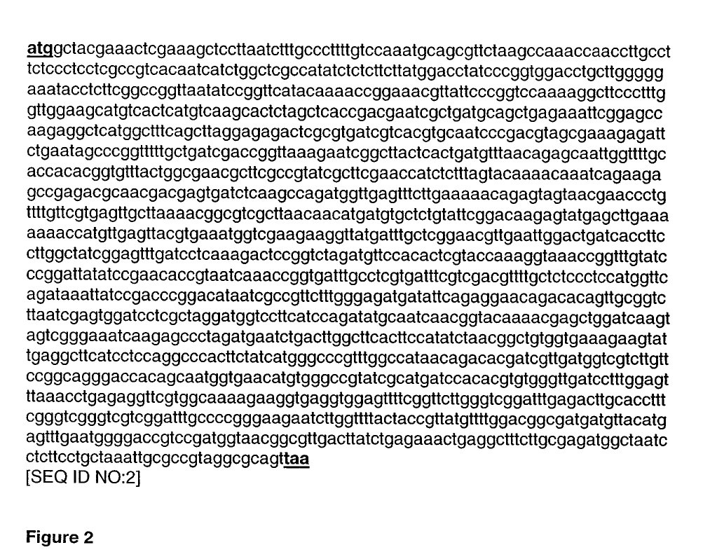

In one variation, the invention includes DNA sequences (and the corresponding polypeptide) including at least one of the sequences shown in FIG. 1 or 2 in a nucleic acid molecule of preferably about: less than 1000 base pairs, less than 1250 base pairs, less than 1500 base pairs, less than 1750 base pairs, less than 2000 base pairs, less than 2250 base pairs, less than 2500 base pairs, less than 2750 base pairs or less than 3000 base pairs.

Regions of the ABACP1 nucleic acid molecule are as follows:

TABLE 1

| | Start cDNA | End cDNA |
|---|---|---|
| | Nucleotide sequence | Nucleotide |
| | [brackets show | sequence [brackets |
| | corresponding amino | show corresponding |
| Nucleic Acid Molecule | acid nos.] | amino acid nos.] |
| Coding region only | 1 (1) | 1590 (530) |
| N-terminal hydrophobic | 52 (18) | 147 (49) |
| Membrane anchoring | | |
| region | | |
| Hinge region Domain | 211 (71) | 228 (76) |
| Heme binding Domain | 1402 (468) | 1431 (477) |

It will be apparent that these may be varied, for example, by shortening the 5′ untranslated region or shortening the nucleic acid molecule so that the 3′ end nucleotide is in a different position.

The discussion of the nucleic acid molecules, sequence identity, hybridization and other aspects of nucleic acid molecules included within the scope of the invention is intended to be applicable to either the entire nucleic acid molecule or its coding region. One may use the entire molecule or only the coding region. Other possible modifications to the sequence are apparent.

The ABACP1 Nucleic Acid Molecule and Polypeptide are Conserved in Plants

Sequence Identity

This is the first isolation of a nucleic acid molecule encoding an ABACP polypeptide from plant species. Nucleic acid sequences having sequence identity to the ABACP1 sequence are found in other plants such as alfalfa, almond, apple, apricot, arabidopsis, artichoke, atriplex, avocado, barley, beet, birch, brassica, cabbage, cacao, cantalope, carnations, castorbean, caulifower, celery, clover, coffee, corn, cotton, cucumber, garlic, grape, grapefruit, hemp, hops, lettuce, maple, melon, mustard, oak, oat, olive, onion, orange, pea, peach, pear, pepper, pine, plum, poplar, potato, prune, radish, rape, rice, roses, rye, sorghum, soybean, spinach, squash, strawberries, sunflower, sweet corn, tobacco, tomato or wheat. We isolate ABACP nucleic acid molecules from the aforementioned plants. The invention includes methods of isolating these nucleic acid molecules and polypeptides as well as methods of using these nucleic acid molecules and polypeptides according to the methods described in this application, for example those methods used with respect to ABACP1.

The invention includes the nucleic acid molecules from other plants as well as methods of obtaining the nucleic acid molecules by, for example, screening a cDNA library or other DNA collections with a probe of the invention (such as a probe comprising at least about: 10 or preferably at least 15 or 30 or more nucleotides of ABACP1 and detecting the presence of an ABACP nucleic acid molecule. Another method involves comparing the ABACP1 sequence to other sequences, for example by using bioinformatics techniques such as database searches or alignment strategies, and detecting the presence of an ABACP nucleic acid molecule or polypeptide. The invention includes the nucleic acid molecule and/or polypeptide obtained according to the methods of the invention. The invention also includes methods of using the nucleic acid molecules, for example to make probes, in research experiments or to transform host cells or make transgenic plants. These methods are as described below.

The polypeptides encoded by the ABACP nucleic acid molecules in other species will have amino acid sequence identity to the ABACP1 sequence. Sequence identity may be at least about: >50% or >55% to an amino acid sequence shown in FIG. 1 or 2 (or a partial sequence thereof). Some polypeptides may have a sequence identity of at least about: >60%, >70%, >80% or >90%, more preferably at least about: >95%, >99% or >99.5% to an amino acid sequence in FIG. 1 or 2 (or a partial sequence thereof). Identity is calculated according to methods known in the art. Sequence identity (nucleic acid and protein) is most preferably assessed by the algorithm of the Fasta 3 program, using the following default parameter settings: gap penalty (open)=−12 (protein)−16 (DNA), gap penalty (extension)=−2 (protein)−4 (DNA), protein weight matrix=BLOSUM 62. (The reference for FASTA 3 is W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA” Methods in Enzymology 183:63-98). The invention also includes modified polypeptide from plants which have sequence identity at least about: >20%, >25%, >28%, >30%, >35%, >40%, >50%, >60%, >70%, >80% or >90% more preferably at least about >95%, >99% or >99.5%, to the ABACP sequence in FIG. 1 or 2 (or a partial sequence thereof). Modified polypeptide molecules are discussed below. Preferably about: 1, 2, 3, 4, 5, 6 to 10, 0 to 25, 26 to 50 or 51 to 100, or 101 to 250 nucleotides or amino acids are modified.

Nucleic Acid Molecules and Polypeptides Similar to ABACP1

Those skilled in the art will recognize that the nucleic acid molecule sequences in FIG. 1 or 2 are not the only sequences which may be used to provide increased ABACP activity in plants. The genetic code is degenerate so other nucleic acid molecules which encode a polypeptide identical to an amino acid sequence in FIG. 1 or 2 may also be used. The sequence of the other nucleic acid molecules of this invention may also be varied without changing the polypeptide encoded by the sequence. Consequently, the nucleic acid molecule constructs described below and in the accompanying examples for the preferred nucleic acid molecules, vectors, and transformants of the invention are merely illustrative and are not intended to limit the scope of the invention.

The sequences of the invention can be prepared according to numerous techniques. The invention is not limited to any particular preparation means. For example, the nucleic acid molecules of the invention can be produced by cDNA cloning, genomic cloning, cDNA synthesis, polymerase chain reaction (PCR), or a combination of these approaches (Current Protocols in Molecular Biology (F. M. Ausbel et al., 1989).). Sequences may be synthesized using well known methods and equipment, such as automated synthesizers. Nucleic acid molecules may be amplified by the polymerase chain reaction. Polypeptides may, for example, be synthesized or produced recombinantly.

Sequence Identity

The invention includes modified nucleic acid molecules with a sequence identity at least about: >17%, >20%, >30%, >40%, >50%, >60%, >70%, >80% or >90% more preferably at least about >95%, >99% or >99.5%, to a DNA sequence in FIG. 1 or 2 (or a partial sequence thereof) and which in a plant are capable of catalyzing the hydroxylation of ABA to 8′hydroxy-ABA. Preferably about 1, 2, 3, 4, 5, 6 to 10, 10 to 25, 26 to 50 or 51 to 100, or 101 to 250 nucleotides or amino acids are modified. Identity is calculated according to methods known in the art. Sequence identity is most preferably assessed by the algorithm of the FASTA 3 program. For example, if a nucleotide sequence (called “Sequence A”) has 90% identity to a portion of the nucleotide sequence in FIG. 1, then Sequence A will be identical to the referenced portion of the nucleotide sequence in FIG. 1, except that Sequence A may include up to 10 point mutations, such as substitutions with other nucleotides, per each 100 nucleotide of the referenced portion of the nucleotide sequence in FIG. 1. Nucleotide sequences functionally equivalent to the ABACP1 sequence can occur in a variety of forms as described below. Polypeptides having sequence identity may be similarly identified.

The polypeptides encoded by the homologous ABACP nucleic acid molecule in other species will have amino acid sequence identity at least about: >20%, >25%, >28%, >30%, >40% or >50% to an amino acid sequence shown in FIG. 1 or 2 (or a partial sequence thereof). Some plant species may have polypeptides with a sequence identity of at least about: >60%, >70%, >80% or >90%, more preferably at least about: >95%, >99% or >99.5% to all or part of an amino acid sequence in FIG. 1 or 2 (or a partial sequence thereof). Identity is calculated according to methods known in the art. Sequence identity is most preferably assessed by the FASTA 3 program. Preferably about: 1, 2, 3, 4, 5, 6 to 10, 10 to 25, 26 to 50 or 51 to 100, or 101 to 250 nucleotides or amino acids are modified.

The invention includes nucleic acid molecules with mutations that cause an amino acid change in a portion of the polypeptide not involved in providing ABACP activity or an amino acid change in a portion of the polypeptide involved in providing ABACP activity so that the mutation increases or decreases the activity of the polypeptide.

Hybridization

Other functional equivalent forms of the ABACP nucleic acid molecules encoding nucleic acids can be isolated using conventional DNA-DNA or DNA-RNA hybridization techniques. These nucleic acid molecules and the ABACP sequences can be modified without significantly affecting their activity.

The present invention also includes nucleic acid molecules that hybridize to one or more of the sequences in FIG. 1 or 2 (or a partial sequence thereof) or their complementary sequences, and that encode peptides or polypeptides exhibiting substantially equivalent activity as that of an ABACP polypeptide produced by the DNA in FIG. 1 or 2 (ie. capable of catalyzing the hydroxylation of ABA to 8′hydroxy-ABA). Such nucleic acid molecules preferably hybridize to all or a portion of ABACP or its complement or all or a portion of an EST of Table 2 under low, moderate (intermediate), or high stringency conditions as defined herein (see Sambrook et al. (Most recent edition) Molecular Cloning: A Laboratory Manual, Cold Spring Harbor Laboratory Press, Cold Spring Harbor, N.Y.; Ausubel et al. (eds.), 1995, Current Protocols in Molecular Biology, (John Wiley & Sons, NY)). The portion of the hybridizing nucleic acids is typically at least 15 (e.g. 20, 25, 30 or 50) nucleotides in length. The hybridizing portion of the hybridizing nucleic acid is at least 80% e.g. at least 95% or at least 98% identical to the sequence or a portion or all of a nucleic acid encoding an ABACP polypeptide, or its complement. Hybridizing nucleic acids of the type described herein can be used, for example, as a cloning probe, a primer (e.g. a PCR primer) or a diagnostic probe. Hybridization of the oligonucleotide probe to a nucleic acid sample typically is performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g. SSC or SSPE). Then, assuming that 1% mismatching results in a 1 degree Celsius decrease in the Tm, the temperature of the final wash in the hybridization reaction is reduced accordingly (for example, if sequences having greater than 95% identity with the probe are sought, the final wash temperature is decreased by 5 degrees Celsius). In practice, the change in Tm can be between 0.5 degrees Celsius and 1.5 degrees Celsius per 1% mismatch. Low stringency conditions involve hybridizing at about: 2×SSC, 0.1% SDS at 50° C. High stringency conditions are: 0.1×SSC, 0.1% SDS at 65° C. Moderate stringency is about IX SSC 0.1% SDS at 60 degrees Celsius. The parameters of salt concentration and temperature can be varied to achieve the optimal level of identity between the probe and the target nucleic acid.

The present invention also includes nucleic acid molecules from any source, whether modified or not, that hybridize to genomic DNA, cDNA, or synthetic DNA molecules that encode the amino acid sequence of an ABACP polypeptide, or genetically degenerate forms, under salt and temperature conditions equivalent to those described in this application, and that code for a peptide, or polypeptide that has ABACP activity. Preferably the polypeptide has the same or similar activity as that of an ABACP polypeptide. A nucleic acid molecule described above is considered to be functionally equivalent to an ABACP nucleic acid molecule (and thereby having ABACP activity) of the present invention if the polypeptide produced by the nucleic acid molecule displays the following characteristic: the defining feature of ABACP polypeptides is the ability to catabolize the conversion of ABA to 8′hydroxy-ABA.

The invention also includes nucleic acid molecules and polypeptides having sequence similarity taking into account conservative amino acid substitutions. Sequence similarity (and preferred percentages) are discussed below.

Modifications to Nucleic Acid Molecule or Polypeptide Sequence

Changes in the nucleotide sequence which result in production of a chemically equivalent or chemically similar amino acid sequences are included within the scope of the invention. Variants of the polypeptides of the invention may occur naturally, for example, by mutation, or may be made, for example, with polypeptide engineering techniques such as site directed mutagenesis, which are well known in the art for substitution of amino acids. For example, a hydrophobic residue, such as glycine can be substituted for another hydrophobic residue such as alanine. An alanine residue may be substituted with a more hydrophobic residue such as leucine, valine or isoleucine. A negatively charged amino acid such as aspartic acid may be substituted for glutamic acid. A positively charged amino acid such as lysine may be substituted for another positively charged amino acid such as arginine.

Therefore, the invention includes polypeptides having conservative changes or substitutions in amino acid sequences. Conservative substitutions insert one or more amino acids which have similar chemical properties as the replaced amino acids. The invention includes sequences where conservative substitutions are made that do not destroy ABACP activity. The preferred percentage of sequence similarity for sequences of the invention includes sequences having at least about: 50% similarity to ABACP1. The similarity may also be at least about: 60% similarity, 75% similarity, 80% similarity, 90% similarity, 95% similarity, 97% similarity, 98% similarity, 99% similarity, or more preferably at least about 99.5% similarity, wherein the polypeptide has ABACP activity. The invention also includes nucleic acid molecules encoding polypeptides, with the polypeptides having at least about: 50% similarity to ABACP1. The similarity may also be at least about: 60% similarity, 75% similarity, 80% similarity, 90% similarity, 95% similarity, 97% similarity, 98% similarity, 99% similarity, or more preferably at least about 99.5% similarity, wherein the polypeptide has ABACP activity, to an amino acid sequence in FIG. 1 or 2 (or a partial sequence thereof) considering conservative amino acid changes, wherein the polypeptide has ABACP activity. Sequence similarity is preferably calculated as the number of similar amino acids in a multiple alignment expressed as a percentage of the shorter of the two sequences in the alignment. The multiple alignment is preferably constructed using the algorithm of the FASTA 3 program, using the following parameter settings: gap penalty (open)=−12(protein)−16 (DNA), gap penalty (extension)=−2 (protein)−4 (DNA), protein weight matrix=BLOSUM 62. (The reference for FASTA 3 is W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA” Methods in Enzymology 183:63-98).

Polypeptides comprising one or more d-amino acids are contemplated within the invention. Also contemplated are polypeptides where one or more amino acids are acetylated at the N-terminus. Those of skill in the art recognize that a variety of techniques are available for constructing polypeptide mimetics with the same or similar desired ABACP activity as the corresponding polypeptide compound of the invention but with more favorable activity than the polypeptide with respect to solubility, stability, and/or susceptibility to hydrolysis and proteolysis. See, for example, Morgan and Gainor, Ann. Rep. Med. Chem., 24:243-252 (1989). Examples of polypeptide mimetics are described in U.S. Pat. No. 5,643,873. Other patents describing how to make and use mimetics include, for example in, U.S. Pat. Nos. 5,786,322, 5,767,075, 5,763,571, 5,753,226, 5,683,983, 5,677,280, 5,672,584, 5,668,110, 5,654,276, 5,643,873. Mimetics of the polypeptides of the invention may also be made according to other techniques known in the art. For example, by treating a polypeptide of the invention with an agent that chemically alters a side group by converting a hydrogen group to another group such as a hydroxy or amino group. Mimetics preferably include sequences that are either entirely made of amino acids or sequences that are hybrids including amino acids and modified amino acids or other organic molecules.

The invention also includes hybrid nucleic acid molecules and polypeptides, for example where a nucleotide sequence from one species of plant is combined with a nucleotide sequence from another sequence of plant, mammal, bacteria or yeast to produce a fusion polypeptide. The invention includes a fusion protein having at least two components, wherein a first component of the fusion protein comprises a polypeptide of the invention, preferably a full length ABACP polypeptide. The second component of the fusion protein preferably comprises a tag, for example GST, an epitope tag or an enzyme. The fusion protein may comprise lacZ.

The invention also includes polypeptide fragments of the polypeptides of the invention which may be used to confer ABACP activity if the fragments retain activity. The invention also includes polypeptides fragments of the polypeptides of the invention which may be used as a research tool to characterize the polypeptide or its activity. Such polypeptides preferably consist of at least 5 amino acids. In preferred embodiments, they may consist of 6 to 10, 11 to 15, 16 to 25, 26 to 50, 51 to 75, 76 to 100 or 101 to 250 amino acids of the polypeptides of the invention (or longer amino acid sequences). The fragments preferably have ABACP activity. Fragments may include sequences with one or more amino acids removed, for example, C-terminus amino acids in an ABACP sequence.

The invention also includes a composition comprising all or part of an isolated ABACP nucleic acid molecule (preferably ABACP1) of the invention and a carrier, preferably in a composition for plant transformation. The invention also includes a composition comprising an isolated ABACP polypeptide (preferably ABACP1) and a carrier, preferably for studying polypeptide activity.

Recombinant Nucleic Acid Molecules

The invention also includes recombinant nucleic acid molecules preferably an ABACP1 sequence of FIG. 1 or 2 comprising a nucleic acid molecule of the invention and a promoter sequence, operatively linked so that the promoter enhances transcription of the nucleic acid molecule in a host cell (the nucleic acid molecules of the invention may be used in an isolated native gene or a chimeric gene, for example, where a nucleic acid molecule coding region is connected to one or more heterologous sequences to form a gene. The promoter sequence is preferably a constitutive promoter sequence or an inducible promoter sequence, operatively linked so that the promoter enhances transcription of the DNA molecule in a host cell. The promoter may be of a type not naturally associated with the cell such as a super promoter, a 35S cauliflower mosaic virus promoter, a chemical inducible promoter, a copper-inducible promoter, a steroid-inducible promoter and a tissue specific promoter.

A recombinant nucleic acid molecule for conferring ABACP activity may also contain suitable transcriptional or translational regulatory elements. Suitable regulatory elements may be derived from a variety of sources, and they may be readily selected by one with ordinary skill in the art. Examples of regulatory elements include: an enhancer or RNA polymerase binding sequence, a ribosomal binding sequence, including a translation initiation signal. Additionally, depending on the vector employed, other genetic elements, such as selectable markers, may be incorporated into the recombinant molecule. Markers facilitate the selection of a transformed host cell. Such markers include genes associated with temperature sensitivity, drug resistance, or enzymes associated with phenotypic characteristics of the host organisms.

Nucleic acid molecule expression levels are controlled with a transcription initiation region that regulates transcription of the nucleic acid molecule or nucleic acid molecule fragment of interest in a plant, bacteria or yeast cell. The transcription initiation region may be part of the construct or the expression vector. The transcription initiation domain or promoter includes an RNA polymerase binding site and an mRNA initiation site. Other regulatory regions that may be used include an enhancer domain and a termination region. A terminator is contemplated as a DNA sequence at the end of a transcriptional unit which signals termination of transcription. These elements are 3′-non-translated sequences containing polyadenylation signals which act to cause the addition of polyadenylate sequences to the 3′ end of primary transcripts. Examples of terminators particularly suitable for use in nucleotide sequences and DNA constructs of the invention include the nopaline synthase polyadenylation signal of Agrobacterium tumefaciens, the 35S polyadenylation signal of CaMV. The regulatory elements described above may be from animal, plant, yeast, bacteria, fungus, virus or other sources, including synthetically produced elements and mutated elements.

Methods of modifying DNA and polypeptides, preparing recombinant nucleic acid molecules and vectors, transformation of cells, expression of polypeptides are known in the art. For guidance, one may consult the following U.S. Pat. Nos. 5,840,537, 5,850,025, 5,858,719, 5,710,018, 5,792,851, 5,851,788, 5,759,788, 5,840,530, 5,789,202, 5,871,983, 5,821,096,5,876,991, 5,422,108, 5,612,191, 5,804,693, 5,847,258, 5,880,328, 5,767,369, 5,756,684, 5,750,652, 5,824,864, 5,763,211, 5,767,375, 5,750,848, 5,859,337, 5,563,246, 5,346,815, and WO9713843. Many of these patents also provide guidance with respect to experimental assays, probes and antibodies, methods, transformation of host cells and regeneration of plants, which are described below. These patents, like all other patents, publications (such as articles and database publications) in this application, are incorporated by reference in their entirety.

Host Cells Including an ABACP Nucleic Acid Molecule

In a preferred embodiment of the invention, a plant or yeast cell is transformed with a nucleic acid molecule of the invention or a fragment of a nucleic acid molecule inserted in a vector.

Another embodiment of the invention relates to a method of transforming a host cell with a nucleic acid molecule of the invention or a fragment of a nucleic acid molecule, inserted in a vector. The invention also includes a vector comprising a nucleic acid molecule of the invention. The nucleic acid molecules can be cloned into a variety of vectors by means that are well known in the art. The recombinant nucleic acid molecule may be inserted at a site in the vector created by restriction enzymes. A number of suitable vectors may be used, including cosmids, plasmids, bacteriophage, baculoviruses and viruses. Suitable vectors are capable of reproducing themselves and transforming a host cell. The invention also relates to a method of expressing polypeptides in the host cells. A nucleic acid molecule of the invention may be used to transform virtually any type of plant, including both monocots and dicots. The expression host may be any cell capable of expressing ABACP, such as a cell selected from the group consisting of a seed (where appropriate), plant cell, bacterium, yeast, fungus, protozoa, algae, animal and animal cell.

Levels of nucleic acid molecule expression may be controlled with nucleic acid molecules or nucleic acid molecule fragments that code for anti-sense RNA inserted in the vectors described above.

Agrobacterium tumefaciens-mediated transformation, particle-bombardment-mediated transformation, direct uptake, microinjection, coprecipitation and electroporation-mediated nucleic acid molecule transfer are useful to transfer an ABACP nucleic acid molecule into seeds (where appropriate) or host cells, preferably plant cells, depending upon the plant species. The invention also includes a method for constructing a host cell capable of expressing a nucleic acid molecule of the invention, the method comprising introducing into said host cell a vector of the invention. The genome of the host cell may or may not also include a functional ABACP gene. The invention also includes a method for expressing an ABACP polypeptide such as an ABACP1 in the host cell or a plant, plant part, seed or plant cell of the invention, the method comprising culturing the host cell under conditions suitable for gene expression. The method preferably also includes recovering the expressed polypeptide from the culture.

The invention includes the host cell comprising the recombinant nucleic acid molecule and vector as well as progeny of the cell. Preferred host cells are fungal cells, yeast cells, bacterial cells, mammalian cells, bird cells, reptile cells, amphibious cells, microorganism cells and plant cells. Host cells may be cultured in conventional nutrient media. The media may be modified as appropriate for inducing promoters, amplifying genes or selecting transformants. The culture conditions, such as temperature, composition and pH will be apparent. After transformation, transform ants may be identified on the basis of a selectable phenotype. A selectable phenotype can be conferred by a selectable marker in the vector.

Transgenic Plants and Seeds

Plant cells are useful to produce tissue cultures, seeds or whole plants. The invention includes a plant, plant part, seed, or progeny of the foregoing, including a host cell transformed with an ABACP nucleic acid molecule such as ABACP1. The plant part is preferably a leaf, a stem, a flower, a root, a seed or a tuber. The transformed plants are useful because they have increased stomate opening and gas exchange. Transformation of seeds also allows control over seed germination. For example, synchronous or early germination may be obtained. In seeds, the gene is preferably expressed under the control of an inducible promoter such as a temperature or chemical sensitive promoter.

The invention includes a transformed (transgenic) plant having increased ABACP activity, the transformed plant containing a nucleic acid molecule sequence encoding for polypeptide activity and the nucleic acid molecule sequence having been introduced into the plant by transformation under conditions whereby the transformed plant expresses an ABACP polypeptide in an active form.

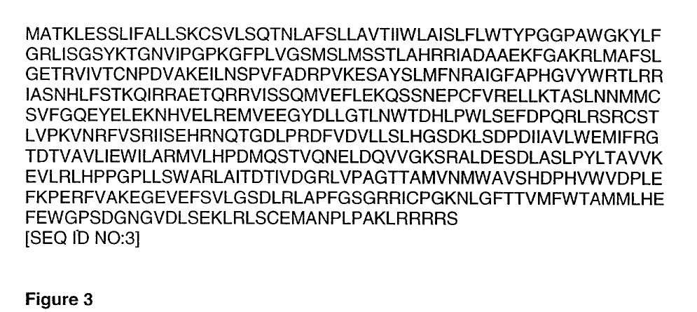

The methods and reagents for producing mature plants from cells are known in the art. The invention includes a method of producing a genetically transformed plant which expresses ABACP polypeptide such as a polypeptide in FIG. 3 by regenerating a genetically transformed plant from the plant cell, seed or plant part of the invention. The invention also includes the transgenic plant produced according to the method. Alternatively, a plant may be transformed with a vector of the invention.

The invention also includes a method of preparing a plant with increased ABACP activity, the method comprising transforming the plant with a nucleic acid molecule which encodes a polypeptide of FIG. 3 or a polypeptide encoding an ABACP polypeptide capable of increasing ABACP activity in a cell, and recovering the transformed plant with increased ABACP activity. The invention also includes a method of preparing a plant with increased ABACP activity, the method comprising transforming a plant cell with a nucleic acid molecule such as a molecule of FIG. 1 or 2 which encodes an ABACP polypeptide capable of increasing ABACP activity in a cell.

Overexpression of ABACP leads to an improved ability of the transgenic plants to catabolize ABA, which can increase gas exchange and help to control seed germination.

The plants whose cells may be transformed with a nucleic acid molecule of this invention and used to produce transgenic plants include, but are not limited to the following: alfalfa, almond, apple, apricot, arabidopsis, artichoke, atriplex, avocado, barley, beet, birch, brassica, cabbage, cacao, cantalope, carnations, castorbean, caulifower, celery, clover, coffee, corn, cotton, cucumber, garlic, grape, grapefruit, hemp, hops, lettuce, maple, melon, mustard, oak, oat, olive, onion, orange, pea, peach, pear, pepper, pine, plum, poplar, potato, prune, radish, rice, roses, rye, sorghum, soybean, spinach, squash, strawberries, sunflower, tobacco, tomato, wheat.

Target plants include: Brassica napus, Brassica rapa, Brassica juncea, Brassica oleracea, or from the family Brassicaecae, Arabidopsis, potato, tomato, tobacco, cotton, carrot, petunia, sunflower, strawberries, spinach, lettuce, rice, soybean, corn, wheat, rye, barley, sorgum and alfalfa. Cereal plants including rye, barley and wheat may also be transformed with an ABACP polypeptide, preferably ABACP1. Other plants listed above are also suitable.

In a preferred embodiment of the invention, plant tissue cells or cultures which demonstrate ABACP activity (or increased ABACP activity compared to wild type) are selected and plants are regenerated from these cultures. Methods of regeneration will be apparent to those skilled in the art (see examples below, also). These plants may be reproduced, for example by cross pollination with a plant that does not have ABACP activity. If the plants are self-pollinated, homozygous progeny may be identified from the seeds of these plants, for example, using genetic markers. Seeds obtained from the mature plants resulting from these crossings may be planted, grown to sexual maturity and cross-pollinated or self-pollinated.

The nucleic acid molecule is also incorporated in some plant species by breeding methods such as back crossing to create plants homozygous for the ABACP nucleic acid molecule.

A plant line homozygous for the ABACP nucleic acid molecule may be used as either a male or female parent in a cross with a plant line lacking the ABACP nucleic acid molecule to produce a hybrid plant line which is uniformly heterozygous for the nucleic acid molecule. Crosses between plant lines homozygous for the ABACP nucleic acid molecule are used to generate hybrid seed homozygous for the resistance nucleic acid molecule.

Antisense and Overexpression Technology

Inhibition of ABACP

To reduce the abundance and thus the activity of the target protein, coding sequences typically obtained from cDNAs are expressed in the reverse orientation in transgenic plants so that the RNA generated is a complement to the endogenous mRNA coding for the target protein. The combination of these two RNAs in planta causes an inability of the target protein mRNA to be translated. Expression of the antisense RNA in planta is usually accomplished using vectors that contain highly active promoter sequences which will produce an abundance of the antisense RNA. A specific example of the use of antisense technology is provided below in “Antisensing and Overexpression Manipulation of cDNA in Wild Type”. Patents that describe generally how to use antisense technology include: U.S. Pat. Nos. 5,859,342, 5,759,829, 5,728,926, 5,684,241, 5,668,295, 5,457,281, 5,453,566, 5,365,015, 5,356,799, 5,316,930, 5,254,800.

The nucleotide sequence encoding the antisense RNA molecule can be of any length providing that the antisense RNA molecule transcribable therefrom is sufficiently long so as to be able to form a complex with a sense mRNA molecule encoding for a polypeptide having ABACP activity in the ABA oxidation pathway. The antisense RNA molecule complexes with the mRNA of the polypeptide and inhibits or reduces the synthesis of ABACP. As a consequence of the interference of the antisense RNA enzyme, the activity of the ABACP polypeptides involved in ABA oxidation is decreased.

The antisense RNA preferably comprises a sequence that is complementary to a portion of the coding sequence for ABACP1, or a portion thereof, or preferably comprises a sequence having at least 20%, 30%, 40%, 50%, 60%, 70%, 80%, 90% or 95% sequence identity to ABACP1 shown in FIG. 1 or 2, or a portion thereof (sequence identity is determined as described above). The sequence may include the 5′-terminus, be downstream from the 5′-terminus, or may cover all or only a portion of the non-coding region, may bridge the non-coding and coding region, be complementary to all or part of the coding region, complementary to the 3′-terminus of the coding region, or complementary to the 3′-untranslated region of the mRNA. The particular site(s) to which the anti-sense sequence binds and the length of the anti-sense sequence will vary, for example, depending upon the degree of inhibition desired, the uniqueness of the sequence and the stability of the anti-sense sequence.

The sequence may be a single sequence or a repetitive sequence having two or more repetitive sequences in tandem, where the single sequence may bind to a plurality of messenger RNAs. In some instances, rather than providing for homoduplexing, heteroduplexing may be employed, where the same sequence may provide for inhibition of a plurality of messenger RNAs by having regions complementary to different messenger RNAs.

The antisense sequence may be complementary to a unique sequence or a repeated sequence, so as to enhance the probability of binding. The antisense sequence may be involved with the binding of a unique sequence, a single unit of a repetitive sequence or of a plurality of units of a repetitive sequence.

The transcriptional construct will preferably include, in the direction of transcription, a transcriptional initiation region, the sequence coding for the antisense RNA on the sense strand, and a transcriptional termination region.

The DNA encoding the antisense RNA can be from about 20 nucleotides in length up to preferably about the length of the relevant mRNA produced by the cell. For example, the length of the DNA encoding the antisense RNA can be from 20 to 1500 or 2000 nucleotides in length. The sequence complementary to a sequence of the messenger RNA will usually be at least about 20, 30, 50, 75 or 100 nucleotides or more, and often being fewer than about 1000 nucleotides. The preferred source of antisense RNA for DNA constructs of the present invention is DNA that is complementary to full length ABACP1, or fragments thereof. DNA showing substantial sequence identity to the complement of ABACP1 or fragments thereof is also useful.

Suitable promoters are described elsewhere in this application and known in the art. The promoter gives rise to the transcription of a sufficient amount of the antisense RNA molecule at a rate sufficient to cause an inhibition or reduction of ABA catabolism in plant cells. The required amount of antisense RNA to be transcribed may vary from plant to plant. Other regulatory elements described in this application, such as enhancers and terminators may also be used. The invention also includes a vector, such as a plasmid or virus including the antisense DNA.

The invention includes the plant cells, for example, the plant cells of the species listed above, containing the antisense sequence. The invention still further provides plants comprising such plant cells, the progeny of such plants which contain the sequence stably incorporated and hereditable, plant parts and/or the seeds of such plants or such progeny.

The invention also includes the use of a sequence according to the invention, in the production of plant cells having a modified ABA content. By “modified ABA content” is meant a cell which exhibits non-wild type proportions of ABA due to inhibited or reduced expression of ABACP.

The invention still further provides a method of inhibiting or reducing expression of an ABACP polypeptide in plant cells, comprising introducing into such cells a nucleic acid molecule according to the invention, such as ABACP1, or a vector containing it. In one example, the invention includes a method for reducing expression of a nucleic acid molecule encoding an ABACP polypeptide, such as ABACP1, comprising: a) integrating into the genome of a plant cell a nucleic acid molecule complementary to all or part of endogenous ABACP mRNA; and b) growing the transformed plant cell, so that the complementary nucleic acid molecule is transcribed and binds to the mRNA, thereby reducing expression of the nucleic acid molecule encoding the ABACP polypeptide. Typically, the amount of RNA transcribed from the complementary strand is less than the amount of the mRNA endogenous to the cell.

The antisense DNA may also comprise a nucleic acid molecule encoding a marker polypeptide, the marker polypeptide also operably linked to a promoter.

Overexpression of ABACP

Overexpression of the target protein is preferably accomplished by transforming plants with a vector containing the ABACP1 DNA in which expression in the normal, forward orientation is now increased by the addition of a highly active promoter to enhance target protein mRNA sysnthesis. Suitable techniques are described above.

Endogenous ABA levels in plants are known to be able to affect the ability of the plant to respond to drought and cold. Increased ABA levels reduce the rate of water loss from the stomate and thus allow the plant to conserve water during periods of low water availability. Changes in endogenous ABA levels are also known to modify plant metabolism such that the plant now exhibits increased ability to tolerate drought and cold conditions.

Increased levels of ABA are involved in the plant sensory apparatus that initates a number of metabolic changes that improve the plant's ability to survive cold and drought Germination of seeds is also affected by the endogenous ABA levels in the seed itself. High levels of ABA repress the ability of the seed to germinate even under optimal conditions. The ability to manipulate ABA levels in planta allows the temporal and spatial control of drought and cold tolerance to enhance these attributes, and allows the temporal control of germination. The pace of the atmospheric CO₂ increase to anticipated levels of 700 ppm by mid century is unprecedented. Elevated CO₂ concentrations can harm photosynthesis of C₃ plants ^{2 3}. For a number of species, the immediate increase in the rate of CO₂ assimilation engendered by increased external CO₂ levels is followed by decline in photosynthetic capacity after prolonged exposure to these same conditions. This acclimation response has been correlated with increases in foliar non-structural carbohydrates, such as hexoses, sucrose, and starch, and is also accompanied by a decline in Rubisco protein levels, transcript abundance for both rbcS and rbcL, as well as a number of other transcripts of proteins required for photosynthesis including chlorophyll a/b binding proteins, carbonic anhydrase, and Rubisco activase ^{45 6}. Reducing ABACP levels in plants is useful for helping plants tolerate a high carbon dioxide environment.

Fragments/Probes

Preferable fragments include 10 to 50, 50 to 100, 100 to 250, 250 to 500, 500 to 1000, 1000 to 1500, or 1500 or more nucleotides of a nucleic acid molecule of the invention. A fragment may be generated by removing a single nucleotide from a sequence in FIG. 1 or 2 (or a partial sequence thereof). Fragments may or may not encode a polypeptide having ABACP activity.

The nucleic acid molecules of the invention (including a fragment of a sequence in FIG. 1 or 2 (or a partial sequence thereof) can be used as probes to detect nucleic acid molecules according to techniques known in the art (for example, see U.S. Pat. Nos. 5,792,851 and 5,851,788). The probes may be used to detect nucleic acid molecules that encode polypeptides similar to the polypeptides of the invention that catabolize ABA. For example, a probe having at least about 10 bases will hybridize to similar sequences under stringent hybridization conditions (Sambrook et al. 1989, Molecular Cloning, A Laboratory Manual, Cold Spring Harbor). Polypeptide fragments of ABACP1 are preferably at least 8 amino acids in length and are useful, for example, as immunogens for raising antibodies that will bind to intact protein (immunogenic fragments). Typically the average length used for synthetic peptides is 8-16, 8 being the minimum, however 12 amino acids is commonly used.

Kits

The invention also includes a kit for conferring increased ABACP activity to a plant or a host cell including a nucleic acid molecule of the invention (preferably in a composition of the invention) and preferably reagents for transforming the plant or host cell.

The invention also includes a kit for detecting the presence of ABACP nucleic acid molecule (e.g. a molecule in FIG. 1 or 2), comprising at least one probe of the invention.

Kits may be prepared according to known techniques, for example, see U.S. Pat. nos. 5,851,788 and 5,750,653.

Antibodies

The invention includes an isolated antibody immunoreactive with a polypeptide of the invention. Antibodies are preferably generated against epitopes of native ABACP1 or synthetic peptides of ABACP1. The antibody may be labeled with a detectable marker or unlabeled. The antibody is preferably a monoclonal antibody or a polyclonal antibody. ABACP antibodies can be employed to screen organisms containing ABACP polypeptides. The antibodies are also valuable for immuno-purification of polypeptides from crude extracts.

Examples of the preparation and use of antibodies are provided in U.S. Pat. Nos. 5,792,851 and 5,759,788. For other examples of methods of the preparation and uses of monoclonal antibodies, see U.S. Pat. Nos. 5,688,681, 5,688,657, 5,683,693, 5,667,781, 5,665,356, 5,591,628, 5,510,241, 5,503,987, 5,501,988, 5,500,345 and 5,496,705. Examples of the preparation and uses of polyclonal antibodies are disclosed in U.S. Pat. Nos. 5,512,282, 4,828,985, 5,225,331 and 5,124,147.

The invention also includes methods of using the antibodies. For example, the invention includes a method for detecting the presence of an ABACP polypeptide such as ABACP1, by: a) contacting a sample containing one or more polypeptides with an antibody of the invention under conditions suitable for the binding of the antibody to polypeptides with which it is specifically reactive; b) separating unbound polypeptides from the antibody; and c) detecting antibody which remains bound to one or more of the polypeptides in the sample.

Research Tool

Cell cultures, seeds, plants and plant parts transformed with a nucleic acid molecule of the invention are useful as research tools. For example, one may obtain a plant cell (or a cell line,) that does not express ABACP, insert an ABACP1 nucleic acid molecule in the cell, and assess the level of ABACP1 expression and activity.

The ABACP nucleic acid molecules and polypeptides including those in the figures are also useful in assays. Assays are useful for identification and development of compounds to inhibit and/or enhance polypeptide function directly.

Suitable assays may be adapted from, for example, U.S. Pat. No. 5,851,788.

Using Exogenous Agents in Combination with a Vector

The nucleic acid molecules of the invention may be used with other nucleic acid molecules that relate to plant protection, for example, nucleic acid molecules that reduce seed germination. Host cells or plants may be transformed with these nucleic acid molecules.

Experiments

A genetic screen was conducted using the small crucifer Arabidopsis thaliana mutagenized by random insertion of T-DNA sequences. 14 day old ambient CO₂ grown plants were screened for their ability to respond differently than wild type plants when exposed to 3000 ppm CO₂ for four days. More specifically, seed mutagenized by the random insertion of T-DNA sequences was surface sterilized, plated, and imbibed at 4° C. for 4 days. Plates were then transferred to ambient CO₂ conditions for 10 days. After 10 days of growth under ambient conditions unhealthy plants were removed from the plates and the plates were then transferred to elevated CO₂ (3000 ppm) conditions for 4 days. The mutant plants were then screened for phenotypes aberrant to wild type.

Two broad categories of mutants were identified; plants that performed better than wild type plants at high levels of CO₂ and were described as CO₂ non-responsive (cnr); and mutants which were affected more than wild type plants by exposure to high levels of CO₂. These mutants were categorized as CO₂ hyper-responsive (chr). The experiments providing isolation and characterization of the CO₂ non-responsive mutant, cnr 2-1, are described below.

Experiment 1: Identification of the T-DNA-Tagged Allele of cnr 2-1.

The mutant cnr 2-1 isolated from the Feldmann T-DNA tagged lines showed a strong insensitive phenotype when grown under high CO₂. Fourteen day old wildtype seedlings grown in constant illumination and exposed to 3000 ppm CO₂ for the four days show significant levels of anthocyanin and the cupping of leaves typically seen in stressed plants. In comparison, the cnr 2-1 plant shows little anthocyanin coloring and no leaf blade deformation. Similar results are seen for plants grown under a 12 hour photoperiod for two weeks and then transferred to high CO₂ conditions for 4 days. Elevated anthocyanin levels and leaf cupping are clearly present in the wild type plants but not in the mutant. In contrast with other high CO₂ insensitive mutants, cnr 2-1 was supersensitive to high levels of exogenous hexoses with little or no germination observed on 5% glucose MS plates. Because of the strong high CO₂ phenotype and the unusual supersensitivity to glucose, this mutant was chosen for further study.

Following isolation of this mutant from the population of tagged lines, a single high CO₂ insensitive plant was selected and allowed to self and the seed from this plant tested for kanamycin resistance and for high CO₂ insensitivity. This process was repeated for 4 generations. Each generation showed 100% resistance to kanamycin and 100% high CO₂ insensitive phenotype. To determine if the lesion was dominant or recessive, an individual plant from the fourth generation of this line was crossed with wild type and the seed (F1 progeny) from five crosses plated and tested for high CO₂ insensitivity. Progeny from all five crosses showed sensitivity to elevated CO₂, indicating that the mutation is a recessive mutation that has caused the high CO₂ insensitive phenotype. F2 progeny segregated 1:3 for kanamycin resistance (kan^{sensitive}/kan^{resistant}) and 3:1 for high CO₂ ^{sensitivity}:high CO₂^{sensitivity}. Of these F2 progeny, 83 high CO₂ insensitive and 21 high CO₂ sensitive plants were selected and placed on MS plates containing 30 μg/ml kanamycin. After 10 days of growth, all 83 high CO₂ insensitive plants displayed 100% kanamycin resistance and all 21 high CO₂ sensitive plants were kanamycin sensitive. On the basis of the number of F2 plant examined, it was concluded that the lesion causing the high CO₂ phenotype was within 20 map units of the T-DNA insert. As this is a large distance, F3 progeny analysis was used to better define the distance between the T-DNA insert and the mutation causing the high CO₂ phenotype. F3 analysis allows the F2 parent genotype to be inferred from the behavior of the F3 progeny on kanamycin containing plates and under high CO₂ conditions. The genotypes of 89 randomly selected F2 parents were determined by F3 progeny analysis. The data show that 45 F2 parents were heterozygous for the kanamycin resistance and for the high CO₂ phenotype. 20 F2 plants were homozygous for the wild type high CO₂ phenotype and all of these generated kanamycin sensitive F3 progeny. 24 F2 parents were found to be both kanamycin resistant and displayed the mutant high CO₂ non-responsive phenotype. No recombinant chromosomes were seen. These data show that the lesion causing the high CO₂ insensitive phenotype) designated as cnr 2-1 is approximately within 1 map unit of the T-DNA insertion.

Experiment 2: Cloning of Genomic Sequence Flanking T-DNA and cDNA

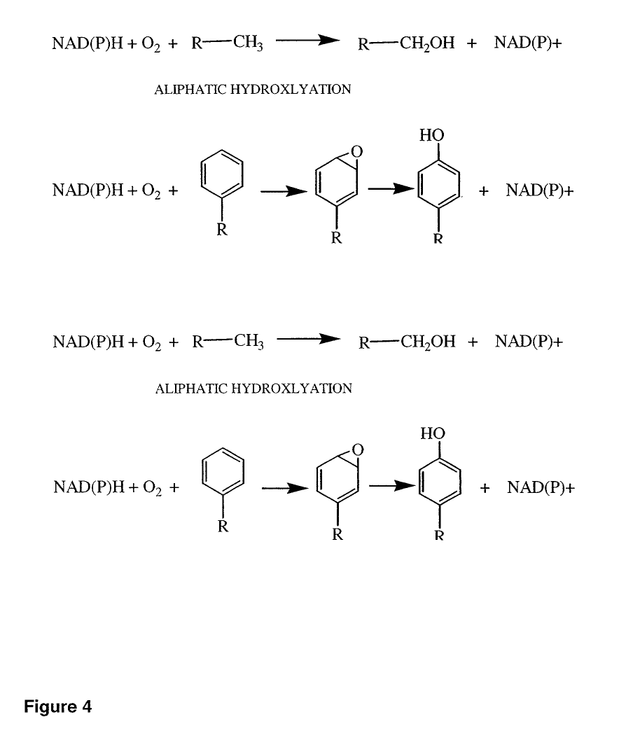

In order to examine the number and structure of inserts in the mutant cnr 2-1, southern blot analysis using the T-DNA right border as a probe of mutant genomic DNA was performed (FIG. 4.3.). The mutant genomic DNA showed three right border insertions when cut with Eco RI. Rather than independent insertion events throughout the genome, these T-DNA border sequences appear to be tandem insertions as the F2 population following crosses with wild type plants segregates 1:3 for kanamycin resistance (kan^{sensitive}: kan^{resistant}). If the insertions were in different chromosomes or different areas of the genome it is likely that at least two of the insertions would segregate and the ratio of kan^{sensitive}:kan^{resistant} plants in the F2 generation would be 1:15. To obtain flanking plant genomic DNA, plasmid rescue was conducted using Sal I and Eco RI digested DNA prepared from the homozygous mutant cnr 2-1. For rescue of plasmids containing left border T-DNA and flanking plant sequences, genomic DNA was digested with Pst I. Five plasmids likely containing plant genomic DNA were identified. These plasmids could be distinguished from sequences containing only T-DNA by the presence of an additional band of plant origin. All five left border plasmids displayed the same Pst I digest pattern. One was selected and designated as 7lb3. To obtain right border plasmids, mutant genomic DNA was digested with Eco RI/SalI, and one plasmid likely containing plant DNA was identified out of nine plasmids recovered. The other eight plasmids appear to contain only T-DNA sequence, identified by the triplet signature of 3.8, 2.4 and 1.2 Kb bands seen on digestion with Eco RI/Sal I. This large proportion of rescued plasmids containing solely T-DNA again suggests tandem right border duplications (FIG. 4.3). The right border plasmid suspected to contain plant DNA was designated.

7rb4 and was sequenced using a pBR322 primer 5′ATTATCACATTAACC3′. This primer is 60 bp away from the EcoRI site on this vector therefore the sequence read using this primer will be plant DNA. The sequence obtained from the right border rescue was 50 bp of plant sequence and part of the NOS terminator. This was deemed insufficient to determine the identity of the site of insertion. The left border rescued plasmid was sequenced using the same pBR322 primer and 460 bp of sequence obtained. Comparison of this sequence with Arabidopsis genomic DNA sequence database showed the left border of the T-DNA insert to be in the 2^{nd} exon of a P450 monooxygenase located on chromosome II. Using a 173 bp Sal I/BamHI fragment from 7lb3 to screen an Arabidopsis cDNA library, a partial cDNA clone was isolated and sequenced. The full-length cDNA was obtained by using gene specific primers and RT PCR. The forward gene specific primer used was (P45F151:5′ TTGATCCGCCATGGCTACGAAACTCG3′), the reverse primer used was (P45R 1976:5′TTAACTGCGCCTACGGCGCAATTTAG3′).

Experiment 3: Sequence Analysis

Blastx analysis indicated that the DNA flanking the insert encodes a cytochrome P450-dependent monoxygenase on chromosome II. Immediately upstream of this P450 open reading frame is a putative cytochrome b5 which might be the electron donor for this P450. BlastP showed most closely related P450s to be CYP78A3 from Glycine max accession # AF022463 (65% identity), a P450 from Pinus radiata accession# AF049067(54% identity), a P450 from Phalaenopsis sp. accession# U34744 (55% identity), and a P450 CYP78 from Zea mays accession # P48420 (48% identity). Many of these CYP78 group P450 monooxygenases had been previously cloned by differential display or subtraction techniques used to obtain inflorescence, tassel and ovule specific genes. The gene structure of the cloned CYP78 is similar to most P450 in that it contains one intron and two exons and belongs to an E class P450 with group I and II signatures.

Experiment 4: Southern and Northern Analysis

To verify that the putative P450 gene was actually disrupted in the cnr 2-1 mutant lines, southern blot analysis of DNA from cnr 2-1 and wild type Arabidopsis ecotype WS was performed. The probe used was a 1.2 Kb EcoRI/NotI cDNA fragment, which spans the T-DNA insert region. The restriction enzymes used for the genomic digest were Eco RI and HindIII as the wild type genomic sequence does contain these restriction sites in the coding region. The T-DNA insertion element, however, does have EcoRI and HindIII restriction sites. The southern clearly show that the region containing the P450 gene to be disrupted by the insertion as two bands are observed for the cnr 2-1 DNA and only one band is observed for the wild type DNA.

Northern blot analysis shows that the CNR2 mRNA is present in plants grown under normal and elevated CO₂ conditions in vegetative tissue. There is a slight increase in transcript abundance under elevated CO₂. No significant levels of hybridization are obtained with RNA isolated from the mutant. Taken together these data show that the cDNA clone identified is disrupted in the cnr 2-1 locus and that the level of expression is extremely low or absent.

To assess the level of CO₂ directed down-regulation of photosynthetic expression in the mutant, the transcript abundance of chlorophyll a/b binding protein (CAB), carbonic anhydrase1 (CA1) and ADP-glucose pyrophosphorylase (ADPGase) was investigated using RNA from 10 day old Arabidopsis wild type seedlings and 10 day old cnr 2-1 seedlings. All plants were grown for six days in air under 200 μmol photons m⁻² s⁻¹ light, plants were then placed in the dark for 4 days. On the fourth day, plants for the air sample were placed in light for 4 hours under ambient CO₂ conditions. Plants for the CO₂ sample were placed in light for 4 hours under 3000 ppm CO₂ conditions. cnr 2-1 showed no change in CAB and CA1 transcript abundance under air or CO₂ conditions, whereas wild type Arabidopsis ecotype WS showed a significant decrease in transcript abundance for both these genes under elevated CO₂ conditions. Furthermore wild type plants showed a significant increase in ADPGase transcripts under high CO₂ conditions whereas transcript levels in cnr 2-1 were only slightly increased under these conditions.

Experiment 5: Physiological Consequences of a Mutation at the cnr 2 Locus

The germination capacity of cnr 2-1 was also investigated. In the absence of chilling, following plating on MS containing agar, the percentage of mutant seed failing to germinate was high compared with wild type seed. Dormancy levels in wild type and cnr 2-1 were therefore measured by chilling seed for increasing amounts of time at 4° C. in darkness. Radicle emergence was measured at 24 hour intervals after imbibition. Chilling increases the percentage and rates of cnr 2-1 germination. The mutant cnr 2-1 seed requires more chilling than the wild type seed and can therefore can be considered to be hyperdormant. When cnr 2-1 seed was plated on 5% glucose MS media, germination was further reduced irrespective of chilling for 4 days. In order to rule out the osmotic effect of high glucose levels in the media, mutant and wild type seed was plated on 5% sorbitol containing MS media. Both wild type and mutant seed exhibited similar germination percentages on the sorbitol containing plates after chilling. The lipid profile and the seed storage proteins of cnr 2 were investigated and found to be similar to wild type showing that differences in seed reserves were not the cause of the reduced germination capacity of the mutant.

As cnr 2-1 was isolated from a high CO₂ screen, a preliminary comparison of CO₂ assimilation response with wild type was conducted. The A/Ci curve for the mutant compared to wild type appears to have a lower initial slope and also a lower saturation point than wild type. Gas exchange analysis also showed that conductance levels for the cnr 2-1 mutant were considerably lower than wild type plants, showing that stomatal responses/aperatures were affected (work in progress by T. Narwani). Scanning electron microscopy of the leaf surface of rapidly killed mutant (OsO₄ fixing) and wild type plants was performed. Preliminary analysis suggests that cnr 2-1 plants have a smaller stomatal aperture than wild type plants.

The cnr 2-1 plants retained more water than the wild type plants after 50 minutes of excision from the root. Kruskal-Wallis one-way analysis of variance on ranks showed data to be significant different after 50 minutes (P=0.001). To further investigate stomatal effects, cnr 2-1 and wild type plants were subjected to dehydration experiments. Dehydration studies of cnr 2-1 and wild type showed that the rate of water loss from wild type plants was higher than the mutant, and that cnr 2-1.

To test for drought tolerance and to determine rates of water loss from rooted plants, the amount of water lost by wild type plants and the cnr2-1 mutant was determined during a drought stress treatment. Five days after withholding water from the plants, pots containing a wild type plant had lost approximately 40% of their initial mass, whereas pots containing an equal biomass of cnr2-1 mutant plants had lost only 33% of their initial mass. After 10 tens drought treatment, pots containing wild type plants had lost 87% of their initial mass whereas cnr2-1 containing pots had lost on 75% of their initial mass. Similar trends were observed over the following 11 days with the cnr2-1 plant continuing to retain more water than wild type plants. At the end of the 21 day period the wild type plants were dry to touch and had lost their turgor whereas the cnr2-l retained turgor with leaves green and flexible (showing drought tolerance).

As the cnr 2-1 mutant displays greater seed dormancy than wild type seed and reduced levels of conductance, water loss following excision of the rosette, and reduced stomatal aperatures as seen in SEM analysis, it was hypothesized that cnr 2-1 might have increased amounts of ABA. Wild type Arabidopsis, cnr 2-1 and the enhanced response to ABA era1-1 seed ¹⁸ were plated on ABA containing MS plates, chilled for three days and allowed to germinate. The cnr 2-1 seed were found to be hypersensitive (reduced germination frequencies) to 0.3 mM ABA in comparison to wild type seed, however cnr 2-1 seed was not as sensitive to exogenous ABA levels as era 1-1 seed. ABA concentrations were also measured in vegetative tissue obtained from well-watered plants and from plants re-watered following 5 days of withholding water.

TABLE 3
Quantification of ABA in fresh and rehydrated tissues.

| | ABA content (picomol/g FW) | | | | | |
|---|---|---|---|---|---|---|
| | Fresh Tissue | | Rehydrated Tissue | | | |
| Genotype | Trial 1 | +/−SD | 1 hour | +/−SD | 5 hours | +/−SD |
| wild type | 227 | 45 | 330 | 76 | 252 | 39 |
| cnr 2-1 | 332 | 21 | 379 | 57 | 358 | 82 |

N = 8 for fresh tissue and N = 24 for rehydrated tissue.

Table 3 shows ABA content to be 40-50% higher in the well-watered cnr 2-1 tissue than in wild type vegetative tissue grown under continuous light and ambient levels of CO₂. One-way ANOVA of ABA content between the mutant and wild type shows that the data are statistically significant (P=0.001, F=35.34) Although re-hydrated tissue show large variations in ABA content, the same trends are observed when cnr 2-1 and wild type are compared. One hour following rehydration, both genotypes show increased amounts of ABA, however after 5 hours the cnr 2-1 ABA level remain high whereas the wild type plant shows a substantial decrease. The data shows that cnr 2-1 plants have similar rates of ABA synthesis but maintain high levels of ABA following water stress treatment.

Materials and Methods

Plant Material

Wild type (Wassilewskija-Ws ecotype) and T-DNA mutagenized Arabidopsis thaliana seed were obtained from the Arabidopsis Biological resource center (ABRC, Ohio State University: stock numbers CS2606-2654). The T-DNA seed collection screened was comprised of 49 pools of 1200 fourth generation offspring derived from 100 mutagenized parents.

Growth Conditions

Seeds were surface sterilized with bleach (10% v/v), rinsed thoroughly and imbibed for 3-5 days at 4° C. prior to sowing in pots containing Pro-Mix or on 0.8% agar supplemented with MS Basal salts (Sigma) buffered at to pH of 5.6 with 50 mM MES (Sigma) under sterile conditions. All plants were grown at 21° C. under continuous illumination of 200 Ξmolm⁻²s⁻¹ PAR or with a 14 h day/10 h night photoperiod where required. The Pro-Mix grown plants were fertilized with 20:20:20 nutrient solution once a week. Plants in pots or on plates were grown in a chamber equipped with an infra-red gas analyser (Horiba) regulator which continuously monitored and maintained the appropriate CO₂ concentration. All molecular and physiological experiments were conducted with plants grown at either ambient (370 ppm) or elevated CO₂ concentrations (1000 ppm) as required.

Genetic Screen

Mutant seed was surface sterilized and imbibed at 4° C. for 4 days, plates were then transferred to ambient conditions for 14 days. After 14 days of growth under ambient conditions unhealthy plants were removed from the plates, and the plates were then transferred to elevated CO₂ conditions for 4 days. The mutant plants were then screened for phenotypes aberrant to wild type.

Genetic Analysis

Mutants were backcrossed to wild type to remove background mutations and to perform segregation analysis. The high CO₂ phenotype of the F1 progeny was examined for all seed from 5 different crosses. Phenotype was analyzed again for the F2 progeny for lack of high CO₂ sensitivity i.e. increased anthocyanin, curling of leaves and necrosis.

Kanamycin Segregation Experiments

To test for linkage of the high CO₂ insensitive mutation in line 87#7 with a T-DNA insertion, a cosegregation experiment using F3 progeny was undertaken. F2 seed from mutants backcrossed to wild type were plated and randomly selected and grown in soil. The F3 seed were harvested from each F2 parent separately and dried for two weeks. After drying, approximately 40 seed from each F3 parent was tested for kanamycin resistance and high CO₂ insensitivity. F2 genotypes were inferred from mutant phenotypes based on the ratio of wild type to mutant seed in each F3 pool tested. CO₂ sensitivity was tested in the same manner as the initial screen and kanamycin sensitivity was measured ten days post-imbibition.

Dormancy Experiments

Dormancy was measured by monitoring germination changes as induced by chilling, (germination was scored by the presence of a radicle). Seed was plated on MS plates and individual plates were chilled for 1 day, 2 days and 3 days at 4° C. Radicle emergence was scored at 24 hour intervals over a 5 day period.

Nucleic Acid Analysis

DNA was isolated from leaf tissue using a method described by Stewart ¹³. Tissue was ground to a powder with liquid nitrogen in a mortar and pestle. The powder was tranferred to a centrifuge tube and 1 ml of 2×CTAB buffer (2% CTAB w/v, 100 mM Tris-HCl pH 8, 20 mM EDTA pH 8, 1.6M NaCl, 1% PVP MW 40000, pre-warmed to 65° C.) was added per gram of fresh weight. 1.5 ml/g FW chloroform: isoamyl alcohol (24:1) was added and mixed thoroughly to form an emulsion. The emulsion was then centrifuged at 10 000 g for 10 min. The upper phase was transferred to a new tube and 1/10 the volume of a 10% CTAB buffer (10%CTAB w/v, 0.7M NaCl pre-warmed to 65° C.) was added and mixed well, the chloroform extraction step was then repeated and after centrifugation the supernatant was transferred to a new tube, to which 1 volume of CTAB precipitation buffer (1% CTAB, 50 mM Tris-HCl pH 8, 10 mM EDTA pH 8) was added. This mixture was allowed to stand overnight at 4° C. The following day the DNA was collected by centrifugation and the DNA pellet was resupended in high salt TE and an appropriate amount of RNAse was added to a final concentration of 100 μg/ml. This was incubated for 1-2 hours at 37° C. Another chloroform extraction step was performed and the supernatant was collected and transferred to a new eppendorf tube after centrifugation. 2 volumes of cold 100% ethanol (stored at −20° C.) were added to the supernatant and the DNA was allowed to precipitate for 15 minutes at −20° C., collected by centrifugation and air-dried for 20-30 minutes. The pellet was rehydrated using 0.1×TE (11.0 mM Tris-HCl pH 8, 0.1 mM EDTA pH 8) to a final concentration of 1 μg/ml.

RNA Isolation

RNA was isolated using the “hot phenol” method ¹⁴. Modifications to this protocol includes addition of a drop of chloroform to overnight precipitation mixture and decanting top layer after centrifugation. DEPC water is added to interphase (containing pellet) and chloroform lower phase. Phenol and chloroform are added to this mixture such that the aqueous phase and organic phase are in a 1:1 ratio. After centrifugation of this mixture, the aqueous top phase is transferred to a new tube and quantified using a UV spectrophotometer. The RNA is distributed into 50 μg aliquots to which 0.1 volumes of 3 M sodium acetate pH 5.2 is added, the RNA is then precipitated with two volumes of ethanol and stored as such.

Southern Analyses

Genomic DNA was cut using restriction enzymes of choice and separated using electrophoresis through 0.8% agarose gels in 0.5×TBE buffer. Gels were soaked in; 0.25M HCl to fragment the DNA; in 0.5M NaOH, 1.5M NaCl to denature DNA and in 1.5 M NaCl, 0.5 M Tris—HCl pH 7.8 neutralization solution. The DNA was then transferred to Nytran (Scheicher and Schuell) by capillary transfer in 20×SSC ¹⁵. Blots were then hybridized with probes synthesized by random priming using the Klenow fragment. Hybridization and washing was carried out using high stringency conditions at 65° C.¹⁵.

Northern Analyses

Formaldehyde gels were used to separate total RNAs using standard protocols ¹⁵. 10-15 μg of RNA was separated in 1.2% formaldehyde agarose gels in 1×MOPS buffer. RNA was transferred to nytran membranes (Schleicher and Schuell) in 20×SSC using capillary action afer soaking the gel in DEPC treated water for 30 minutes. Blots were probed with radiolabelled DNA hybridized in 5% dextran sulphate solution. Washes were done under stringent conditions as per standard protocols. Northerns for P450 transcript level were probed with a 1.2 Kb Eco RI/Not I fragment of the cDNA clone 3-3a.

Plasmid Rescue

Plasmid rescue was performed as described by Dilkes ¹⁶. DNA was isolated from the cnr 2-1 mutant and digested with Eco RI and Sal 1 for right border and left border rescues, respectively. Five μg of DNA was incubated with 125 units of T4 ligase at 16° C. overnight in a total volume of 500 μl. The ligation mixture was phenol: chloroform extracted and concentrated by precipitation. The concentrated mixture was electroporated into competent DH5-α cells. Cells were then plated on 50 μg/ml ampicillin LB plates.

Identification of cDNA and Genomic Clones

A specific 173 bp Sal I/BamHI genomic fragment from the plasmid rescue #71b3 was used to screen an Arabidopsis cDNA library, PRL2 obtained from the ABRC(stock # CD4-7). The PRL library is constructed in Lambda ZipLox, which allows for the automatic excision of the cDNA inserts into plasmid forms. After the tertiary screen three different sized clones were isolated from approximately 200 000 plaques. The biggest cDNA isolated was 1.2 Kb. On sequencing this clone was shown to contain the 2^{nd} exon and part of the 1^{st} exon. The full-length cDNA was obtained by RT-PCR (Statagene) using poly T RNA as a template and gene specific primers. The resultant product was cloned into pGEM T-easy (Promega) and pPCR-Script (Stratagene) vectors.

Antisensing and Overexpression Manipulation of cDNA in Wild Type

Although, F3 analysis strongly suggests that the T-DNA insertion is within approximately 1 map unit from cnr 2-1, it does not prove that the cnr 2-1 mutation is caused by a T-DNA insertion disruption of the CYP 78 or CNR2. To demonstrate that the CNR2 causes the CO₂ non-responsive phenotype, a number of constructs were made using binary vectors and the full length cDNA of CYP78 (CNR2). The full-length cDNA was amplified by PCR using forward primer KpnI/EcoRI P450F (5′-3′: GGGTACCGAATTCATGGCTACGAAACTCGAAAGC) and reverse primer HindIII/SacI P450R (GCATAAGCTTGAGCTCTTAACTGCGCCTACGGCGCA). The amplification conditions were as follows: a single denaturing step at 94° C. for two minutes preceded the 30 cycles of 30 seconds at 94° C.; 60 seconds at 60° C.; and a final elongation step at 72° C. for 90 seconds. The resultant amplification product was cloned into pGEM-T-EASY (Promega). The overexpression and antisense constructs were made in the following manner. The HindIII/XbaI fragment containing the 35S CaMV promoter from pBI221 was cloned into the respective sites in pBS (pBS-35S). For the anti-sense orientation, the CNR2 amplification product was digested with SacI and Eco RI and ligated into the respective sites in pBS-35S to create pCNR2-AS. For the over-expression orientation, the CNR2 amplification product was digested with SacI and KpnI and inserted into the respective sites of pBS-35S to generate pCNR2-OV. To facilitate the insertion of the above constructs into a binary vector, pGPTV-ZERO was fitted with the pZERO-1 (Invitrogen) polylinker using HindIII and XbaI to generate pGPTV-ZERO. CNR2-AS was cloned into pGPTV-Kan as a HindIII/SacI fragment. CNR2-OV was cloned into pGPTV-ZERO as a HindIII/EcoRI. Fragment. To examine the cellular localization of CNR2, another construct was made in pEGAD (a gift from S. Cutler) where the CNR2 amplification product was cloned in frame with the GFP downstream of the alanine flexi-linker region into the Eco RI/HindIII cloning sites. Wild type Arabidopsis WS plants were transformed with the antisense construct, the overexpression construct and the pEGAD constructs ¹⁷.

Carbohydrate and Pigment Analysis

Extraction of Soluble Sugars

Previously frozen and dried plant material was ground to a powder. 15 mg of this plant material was extracted with 2 ml of a solvent mixture of methanol, chloroform and water in a ratio of 12:5:3. The mixture was vortexed and incubated for 20 minutes then later centrifuged to pellet the insoluble material. The supernatant was then removed and placed in a 13 ml snap-cap tube on ice. This extraction procedure of the pellet was then repeated twice. After the final extraction, 2 ml of distilled water was added to the 6 ml of collected supernatant, vortexed and placed at 4° C. overnight. The following day, 200%l of the aqueous upper phase containing the soluble sugars was assayed for soluble sugar content.

Starch Extraction

The remaining pellet after the extraction of soluble sugars was dried overnight in a fume hood and later digested for 1 hour with 35% perchloric acid (v/v), in order to convert polysaccharides into monosaccharides. The mixture was then filtered (standard laboratory glass-fibre filter GFA, Machery-Nagel) and the supernatant was assayed for soluble sugar content.

Assay for Reducing Sugars

200 μl of the starch or soluble sugar solutions extracted by methods described above were placed in 13 ml tubes, 800 μl of water and 1 ml of phenol (5% aqueous w/w) was added to the sample. The mixture was agitated and a stream of 5 ml of concentrated sulphuric acid was delivered by pipette into the mixture. The solution was incubated at 37° C. for 5 minutes for color development and the absorbance was measured at 490 nm in a spectrophotomer. This absorbance was compared with a standard curve using glucose solutions of known concentrations.

Chlorophyll Assay

Portions (0.1 g, FW-fresh weight) of previously weighed foliar tissue was frozen and ground to a fine powder in liquid nitrogen. Thereafter, 80% (v/v) buffered acetone (containing 2.5 mM sodium phosphate pH 7.8) was added to the pulverized tissue (1 ml/100 mg of fresh weight) and the mixture was vortexed twice and centrifuged for 10 minutes at 10 000×g at 4° C. The supernatant was assayed for chlorophyll by measuring absorbance at 645 and 663 nm. Chlorophyll content was calculated using the standard formula, Ch1(a+b)μg/ml=A₆₄₅(20.2)+A₆₆₃ (8.02).

Anthocyanin Assay

Portions (0.5 g) of previously weighed and frozen tissue was ground to a fine powder in liquid nitrogen and the tissue extracted with 1.0 ml of acidic methanol (95% methanol containing 0.1M HCl) by incubating the tissue in the acidic methanol for 16 hours at room temperature. The following day the mixture was centrifuged for 15 minutes at 10 000×g and the anthocyanin content of the supernatant measured spectrophotometrically by determining absorbance at 530 nm and 657 nm. The amount of anthocyanin in relative units is calculated by subtracting absorbance at 657 nm from absorbance at 530 nm.

Sequence Analysis

Sequence analyses were performed using BLASTX and DNASIS (Hitachi).

Plant Transformation

Arabidopsis plants were transformed as described in Desfeux et al, Plant Physiology, Volume 123, p895-904 2000. Aerial portions of plants containing secondary bolts of 1-10 cm in length with multiple young floral buds were dipped for a few seconds into a 300 mls of solution containing 5% (w/v) sucrose, 10 mM MgCl₂ resuspended Agrobacterium cells transformed with the appropriate T-DNA containing vector, and 0.03% (v/v) Silwet L-77 surfactant. After dipping the plants were covered with plastic to maintained humidity and placed in low light conditions for 12-24 hours. Plants were then moved to normal growth conditions and were allowed to set seed. Transformed seed was selected by plating seed out on kanamycin-containing plates and identifying individuals that survived.

Lipid Analysis

Seed (50) were placed in a 50 ml screw capped tube. Three wild type and three cnr 2-1 samples were extracted. 1 ml of HCl(1.5N):CH3OH (dry) was placed in the tube with the seed. This incubation with acidic methanol results in the formation of methanolic esters of fatty acids present in the sample. The mixture was microwaved for 2 minutes and allowed to cool down and vortexed, this was repeated twice more for 1 minute intervals. If the sample lost volume while microwaving more HCl(1.5N):CH₃OH (dry) was added to keep the volume approximately constant. A known amount of 15:0 fatty acid was added to the sample as a standard. 0.5 ml of water and 1 ml of hexane was added to the tube, vortexed vigorously for 2-3 minutes, and later centrifuged for 10 minutes at 2000 rpm. 1 ml of the top fraction was extracted and dryed using nitrogen gas. The sample was then resuspended in 200 μlof hexane and loaded onto a lipid column for GC for analysis.

Determination of ABA Content

40 mg of leaf tissue for plants grown under continuous light conditions was frozen in liquid nitrogen. The leaf tissue was then powdered and 400 μl of 80% acetone was added. The tissue was then incubated in the acetone at 4° C. for 24 hours in the dark. After this extraction procedure, the mixture was centrifuged and the supernatant was removed and diluted 1:50 in PBS and used for ELISAs (Phytodetek ABA, agdia Inc.). Microtitre wells are coated with a monoclonal antibody to ABA and ELISA uses the competitive antibody binding method to measure concentrations of ABA in the plant extract. 100 μl ABA labeled with alkaline phophatase (tracer) is added to wells along with 100 μl plant extract or standard to each ELISA microtitre well. A competitive binding reaction is set up in the sample between constant amount of tracer, a limited amount of antibody and the sample containing an unknown amount of ABA. The hormone in the sample competes with the tracer for antibody binding sites. After 3 hours of incubation at 4° C., the tracer is washed away three times using a wash buffer. A substrate for the alkaline phophatase conjugate was added and incubated for 1 hour at 37° C. A stop solution (1M NaOH) was then added after the incubation. Color absorbance at 405 nm was measured after 5 minutes using a dynatech MR700 plate reader. Each sample for the ABA measurements was taken from a fully expanded leaf. Each trial consisted of 4 plants for each genotype. Duplicates for samples and standards were included on every plate. One-way analysis of variance (ANOVA) showed that trial 1 and 2 should be pooled.

Dehydration Assay

A crude assay to measure dehydration was carried out on wild type and cnr 2-1 plants of comparable size and weight. 3-week old plants were excised at the root and fresh weight of the rosette leaves was measured at 20-minute intervals. The loss of water was measured as a percentage of the plants initial weight. Five plants were used for each genotype. One-way ANOVA analysis was performed between wild type and mutant data for each point in time.

Drought Tolerance Assay

Wild type and cnr2-1 plants were germinated on agar plates and single plants were transferred to pots containing soil where they were allowed to grow for 2 weeks under well watered conditions and a 10/14 light/dark light regime and at 21 C. All soil surfaces were covered with foil to eliminate non-plant mediated water loss. Following two weeks growth, pots containing a single well watered plant of equal size for both geneotypes were then weighed and returned to the growth environment and drought stressed by withholding water for the following 21 days. Pots were weighed daily and the decline in mass attributed to water loss by transpiration calculated as a percentage of the initial weight.

The present invention has been described in detail and with particular reference to the preferred embodiments; however, it will be understood by one having ordinary skill in the art that changes can be made thereto without departing from the spirit and scope of the invention.

All articles, patents and other documents described in this application (including database sequences and/or accession numbers) are incorporated by reference in their entirety to the same extent as if each individual publication, patent or document was specifically and individually indicated to be incorporated by reference in its entirety. They are also incorporated to the extent that they supplement, explain, provide a background for, or teach methodology, techniques and/or compositions employed herein.

### REFERENCES

1. Jouzel, J., Barkov, N. I., Bamola, J. M., Bender, M., Chapellaz, J., Genthon, C., Kotlyakov, V. M., Lipenkov, V., Lorius, C., Petit, J. R., Raynaud, D., Raisbeck, G., Ritz, C., Stievenard, M., Yiou, F., and Yiou, P. Extending the Vostock ice-core record of paleoclimate to the penultimate glacial period. Nature 364, 407-412 (1993).

2. Stitt, M. Rising CO₂ levels and their potential significance for carbon flow in photosynthetic cells. Plant Cell and the Environment 14, 741-762 (1991).

3. Sage, R. F., Reid C. T. in “Plant Response Mechanisms to the Environment”. 413-499 (ed. Wilkinson, R. E., 1994).

4. Van Ooosten, J. J., Besford R. T. Sugar feeding mimics effect of acclimation to high CO₂-rapid down regulation of Rubisco small subunit transcripts but not of the large subunit transcripts. Journal of Plant Physiology 143, 306-312 (1994).

5. Majeau, N., Coleman J. R. Effects of CO₂ concentration on carbonic anhydrase and ribulose 1,5-bisphosphate carboxylase/oxygenase expression in pea. Plant Physiology 112, 569-574 (1996).

6. Cheng, S. H., Moore, B. & Seemann, J. R. Effects of short- and long-term elevated CO₂ on the expression of ribulose-1,5-bisphosphate carboxylase/oxygenase genes and carbohydrate accumulation in leaves of Arabidopsis thaliana (L.) Heynh. Plant Physiol 116, 715-23 (1998).

7. Sheen, J. Metabolic repression of transcription in higher plants. Plant Cell 2, 1027-38 (1990).

8. Krapp, A., Hofmann, B., Schafer, C., Stitt, M. Regulation of the Expression of RBCs and other Photosynthetic Genes by Carbohydrates. A Mechanism for the Sink Regulation of Photosynthesis. Plant Journal 3, 817-828 (1993).

9. Jang, J. C. & Sheen, J. Sugar sensing in higher plants. Plant Cell 6, 1665-79 (1994).

10. Smeekens, S. Sugar regulation of gene expression in plants. Curr Opin Plant Biol 1,230-4 (1998).

11. Halford, N., Purcell, P., Hardie, G. Is hexokinase really a sugar sensor in plants? Trends in Plants Science 4, 117-119 (1999).

12. Lalonde, S., Boles, E., Hellmann, H., Barker, L., Patrick, J. W., Frommer, W. B. and Ward, J. The dual Function of Sugar Carriers: Transport and sugar Sensing. Plant Cell 11, 707-726 (1999).

13. Stewart, C. N., Jr. & Via, L. E. A rapid CTAB DNA isolation technique useful for RAPD fingerprinting and other PCR applications. Biotechniques 14, 748-50 (1993).

14. Verwoerd, T. C., Dekker, B. M. & Hoekema, A. A small-scale procedure for the rapid isolation of plant RNAs. Nucleic Acids Res 17, 2362 (1989).

15. Sambrook, J., Fritsch, E. F., and Maniatis, T. Molecular Cloning: A Laboratory Manual (Cold Spring Harbour Laboratory Press, Cold Spring Harbour, 1989).

16. Dilkes, B. P. & Feldmann, K. A. Cloning genes from T-DNA tagged mutants. Methods Mol Biol 82, 339-51 (1998).

17. Schultz, B., Bennet, M. J., Dilkes, B. P., and Feldmann K. A. T-DNA tagging in Arabidopsis thaliana: cloning by gene disruption (1994).

18. Cutler, S., Ghassemian, M., Bonetta, D., Cooney, S., McCourt, P. A protein farnesyl transferase involved in abscisic acid signal transduction in Arabidopsis. Science 273, 1239-1241 (1996).

19. Degttyarenko, K. N., Archakov, A. I. Molecular Evolution of P450 Superfamily and P450-containing Monooxygenase systems. FEBs 332, 1-8 (1993).

20. Nelson, D. R. Cytochrome P450 and the Individuality of species. Archives of Biochemistry and Biophysics 369, 1-10 (1999).

21. Leymarie, J., Lasceve, G., and Vavasseur, A. Elevated CO₂ enhances stomatal responses to osmotic stress and absicisic acid in Arabidopsis thaliana. Plant, Cell and Environment 22, 301-308 (1999).

22. Krochko, J. E., Abrams, G. D., Loewen, M. K., Abrams, S. R. & Cutler, A. J. (+)-Abscisic acid 8′-hydroxylase is a cytochrome P450 monooxygenase. Plant Physiol 118, 849-60 (1998).

23. Cutler, A. J. & Krochko, J. E. Formation and breakdown of ABA. Trends Plant Sci 4,472-478 (1999).

We claim:

1. An isolated nucleic acid molecule encoding an ABACP polypeptide, or a fragment of an ABACP polypeptide having ABACP polypeptide activity.

2. The molecule of claim 1, wherein the polypeptide catabolizes ABA.

3. The molecule of claim 2, wherein the polypeptide comprises a (+)-ABA 8′hydroxylase.

4. An isolated nucleic acid molecule encoding an ABACP polypeptide, a fragment of an ABACP polypeptide having ABACP activity, or a polypeptide having ABACP activity, comprising a nucleic acid molecule selected from the group consisting of:
a) a nucleic acid molecule that hybridizes to a nucleic acid molecule consisting of [SEQ ID NO:1 or 2], or a complement thereof under low, moderate or high stringency hybridization conditions wherein the nucleic acid molecule encodes an ABACP polypeptide or a polypeptide having ABACP activity;
b) a nucleic acid molecule degenerate with respect to (a), wherein the nucleic molecule encodes an ABACP polypeptide or a polypeptide having ABACP activity.

5. The nucleic acid molecule of claim 4, wherein the hybridization conditions comprise low stringency conditions of 1×SSC, 0.1% SDS at 50° C. or high stringency conditions of 0.1×SSC, 0.1% SDS at 65° C.

6. An isolated nucleic acid molecule encoding an ABACP polypeptide, a fragment of an ABACP polypeptide having ABACP activity, or a polypeptide having ABACP activity, comprising a nucleic acid molecule selected from the group consisting of:
a) the nucleic acid molecule of the coding strand shown in [SEQ ID NO:1 or 2], or a complement thereof;
b) a nucleic acid molecule encoding the same amino acid sequence as a nucleotide sequence of (a); and
c) a nucleic acid molecule having at least 17% identity with the nucleotide sequence of (a) and which encodes an ABACP polypeptide or a polypeptide having ABACP activity.

7. The nucleic acid molecule of claim 1, wherein the ABACP polypeptide comprises a (+)-ABA 8′hydroxylase polypeptide.

8. The nucleic acid molecule of claim 1, comprising all or part of a nucleotide sequence shown in [SEQ ID NO:1 or 2] or a complement thereof.

9. The nucleic acid molecule of claim 1, consisting of the nucleotide sequence shown in [SEQ ID NO:1 or 2] or a complement thereof.

10. The nucleic acid molecule of claim 1, wherein the molecule comprises genomic DNA, cDNA or RNA.

11. The nucleic acid molecule of claim 1, wherein the nucleic acid molecule is chemically synthesized.

12. The nucleic acid molecule of claim 1, comprising at least 30 consecutive nucleotides of [SEQ ID NO:1 or 2] or a complement thereof.

13. A host cell comprising the recombinant nucleic acid molecule of claim 1, or progeny of the host cell.

14. The host cell of claim 13, selected from the group consisting of a fungal cell, a yeast cell, a bacterial cell, a microorganism cell and a plant cell.

15. A plant, a plant part, a seed, a plant cell or progeny thereof comprising the nucleic acid molecule of claim 1.

16. The plant part of claim 15, comprising all or part of a leaf, a flower, a stem, a root or a tuber.

17. The plant, plant part, seed or plant cell of claim 15 wherein the plant, plant part, seed or plant cell is of a species selected from the group consisting of alfalfa, almond, apple, apricot, arabidopsis, artichoke, atriplex, avocado, barley, beet, birch, brassica, cabbage, cacao, cantalope, carnations, castorbean, caulifower, celery, clover, coffee, corn, cotton, cucumber, garlic, grape, grapefruit, hemp, hops, lettuce, maple, melon, mustard, oak, oat, olive, onion, orange, pea, peach, pear, pepper, pine, plum, poplar, potato, prune, radish, rice, roses, rye, sorghum, soybean, spinach, squash, strawberries, sunflower, tobacco, tomato, wheat.

18. The plant, plant part, seed or plant cell of claim 15, wherein the plant comprises a dicot plant.

19. The plant, plant part, seed or plant cell of claim 15, wherein the plant comprises a monocot plant.
